(19) United States
(12) Patent Application Publication
(10) Pub. No.: US 2026/0191610 A1
(43) Pub. Date: Jul. 9, 2026

(54) OBJECT DETECTION AND VISUAL FEEDBACK SYSTEM

(71) Applicant: INTUITIVE SURGICAL OPERATIONS, INC., Sunnyvale, CA (US)

(72) Inventors: Andreas Fatschel, San Francisco, CA (US); Ehab Al Khatib, Santa Clara, CA (US); Amir Chaghajerdi, Los Altos, CA (US); Lawton N. Verner, Saratoga, CA (US)

(21) Appl. No.: 19/130,885

(22) PCT Filed: Nov. 17, 2023
(86) PCT No.: PCT/US2023/080316

§ 371 Date: May 16, 2025

Related U.S. Application Data
(60) Provisional application No. 63/426,594, filed on Nov. 18, 2022.

(51) Int. Cl. A61B 34/37; A61B 34/00; A61B 34/20; G08B 3/10

(52) U.S. Cl. CPC A61B 34/37; A61B 34/76; A61B 2034/2055; A61B 2034/2065; G08B 3/10

(57) ABSTRACT

An object detection and visual feedback system comprises a button with a selection surface positioned for selection by an object. A range sensor has a field of view across the selection surface of the button. The range sensor measures a distance of an object within the field of view. A user interface performs a first action upon the measured distance being less than or equal to a first threshold distance. The user interface performs a second action upon the measured distance being at least a second threshold distance. The second threshold distance is greater than the first threshold distance. The first threshold distance is at an edge of the button. The second threshold distance is not coextensive with the button and spaced apart from the edge of the button by a predetermined distance. The predetermined distance is sufficient to mitigate against accidental selection of the button by the object.

### CROSS-REFERENCE TO RELATED APPLICATIONS

This application claims priority to and the benefit of U.S. Provisional Patent App. No. 63/426,594, filed Nov. 18, 2022, which is incorporated herein by reference in its entirety.

### BACKGROUND

Minimally invasive medical techniques are intended to reduce the amount of extraneous tissue that is damaged during diagnostic or surgical procedures, thereby reducing patient recovery time, discomfort, and harmful side effects. Such minimally invasive techniques may be performed through natural orifices in a patient anatomy or through one or more surgical incisions. Through these natural orifices or incisions, clinicians may insert medical tools to reach a target tissue location. Minimally invasive medical tools include instruments such as therapeutic instruments, diagnostic instruments, and surgical instruments. Minimally invasive medical tools may also include imaging instruments such as endoscopic instruments that provide a user with a field of view within the patient's anatomy.

Some minimally invasive medical tools may be robot-assisted including teleoperated, remotely operated, or otherwise computer-assisted. During a medical procedure, the clinician may be provided with a graphical user interface including an image of a three-dimensional field of view of the patient anatomy. To improve the clinician's experience and efficiency, various indicators may be needed to provide additional information about medical tools in the field of view, medical tools occluded in the field of view, and components outside of the field of view.

### SUMMARY

A first aspect of the disclosure includes an object detection and visual feedback system. The system comprises a button positioned for selection by an object. The button has a selection surface. The system comprises a range sensor with a field of view across the selection surface of the button. The range sensor is configured to measure a distance of an object within the field of view. The system comprises a user interface configured to perform a first action upon the distance measured by the range sensor being less than or equal to a first threshold distance. The user interface is further configured to perform a second action upon the distance measured by the range sensor being greater than or equal to a second threshold distance.

In some implementations of the first aspect of the disclosure, the second threshold distance is greater than the first threshold distance.

In any of the above implementations of the first aspect of the disclosure the first threshold distance is at an edge of the button.

In any of the above implementations of the first aspect of the disclosure the second threshold distance is not coextensive with the button and spaced apart from the edge of the button by a predetermined distance.

In any of the above implementations of the first aspect of the disclosure the predetermined distance is sufficient to mitigate against accidental selection of the button by the object.

In any of the above implementations of the first aspect of the disclosure the predetermined distance is in a range of distances from 10 mm to 30 mm.

In any of the above implementations of the first aspect of the disclosure the object is any one of a non-hand limb, a foot, a leg, a knee, a head, an elbow, or an extension from human anatomy.

In any of the above implementations of the first aspect of the disclosure the range sensor is any of a time-of-flight distance sensor, a triangulation distance sensor, an optical distance sensor, an acoustic distance sensor, an inductive distance sensor, a capacitive distance sensor, a photoelectric distance sensor, a camera, an infrared distance sensor, a laser range finder, or a light detection and ranging sensor.

In any of the above implementations of the first aspect of the disclosure a direction of the field of view is parallel to a direction of the distance measured by the range sensor.

In any of the above implementations of the first aspect of the disclosure a direction of the field of view is orthogonal to a direction of the distance measured by the range sensor.

In any of the above implementations of the first aspect of the disclosure, the system further comprises a second button positioned for selection by an object. The second button has a second selection surface. The system further comprises a second range sensor with a second field of view across the second selection surface of the second button. The second range sensor is configured to measure a distance of an object within the second field of view. The user interface is configured to perform a third action upon the distance measured by the second range sensor being less than or equal to a third threshold distance.

In any of the above implementations of the first aspect of the disclosure the third action is different than the first action.

In any of the above implementations of the first aspect of the disclosure the first action is to display an indication of the button.

In any of the above implementations of the first aspect of the disclosure the second action is to discontinue display of the indication of the button.

In any of the above implementations of the first aspect of the disclosure the indication of the button is selected from a group of indications consisting of an indication of a function performed upon selection of the button; a change of intensity of an icon displayed on the user interface; and a graphic of the object relative to a layout of selection buttons, the layout including the button.

In any of the above implementations of the first aspect of the disclosure the first action is to sound a first audible alert and the second action is to sound a second audible alert.

In any of the above implementations of the first aspect of the disclosure the first action is to provide a first haptic feedback and the second action is to provide a second haptic feedback.

In any of the above implementations of the first aspect of the disclosure the first haptic feedback and the second haptic feedback is provided to a hand controller.

A second aspect of the disclosure includes a robotic surgical system. the system comprises a user interface and a foot tray. The foot tray comprises a button positioned for selection by a foot of a user. The button has a selection surface. The foot tray also comprises a range sensor positioned with a field of view across the selection surface of the button. The range sensor is configured to measure a distance of an object within the field of view. The user interface is configured to perform a first action associated with the button upon the distance measured by the range sensor being less than or equal to a first threshold distance. The user interface is further configured to perform a second action associated with the button upon the distance measured by the range sensor being greater than or equal to a second threshold distance.

In some implementations of the second aspect of the disclosure, the second threshold distance is greater than the first threshold distance.

In any of the above implementations of the second aspect of the disclosure the foot tray is positioned on a base of the robotic surgical system.

In any of the above implementations of the second aspect of the disclosure the first threshold distance is at or near an edge of the button.

In any of the above implementations of the second aspect of the disclosure the second threshold distance is not coextensive with the button and spaced apart from the edge of the button by a predetermined distance.

In any of the above implementations of the second aspect of the disclosure the predetermined distance is sufficient to mitigate against accidental selection of the button by the foot of the user.

In any of the above implementations of the second aspect of the disclosure the predetermined distance is in a range of distances from 10 mm to 30 mm.

In any of the above implementations of the second aspect of the disclosure the range sensor is any of a time-of-flight distance sensor, a triangulation distance sensor, an optical distance sensor, an acoustic distance sensor, an inductive distance sensor, a capacitive distance sensor, a photoelectric distance sensor, a camera, an infrared distance sensor, a laser range finder, or a light detection and ranging sensor.

In any of the above implementations of the second aspect of the disclosure a direction of the field of view is parallel to a direction of the distance measured by the range sensor.

In any of the above implementations of the second aspect of the disclosure a direction of the field of view is orthogonal to a direction of the distance measured by the range sensor.

In any of the above implementations of the second aspect of the disclosure the foot tray further comprises a second button positioned for selection by a foot of a user. The second button comprising a second selection surface. The foot tray further comprises a second range sensor positioned with a second field of view across the second selection surface of the second button. The second range sensor is configured to measure a second distance of an object within the second field of view. The user interface is further configured to perform a third action associated with the second button upon the second distance measured by the second range sensor being less than or equal to a third threshold distance.

In any of the above implementations of the second aspect of the disclosure the third action is different than the first action.

In any of the above implementations of the second aspect of the disclosure the user interface comprises a display.

In any of the above implementations of the second aspect of the disclosure, the system further comprises a head rest, wherein the display is incorporated into the head rest.

In any of the above implementations of the second aspect of the disclosure the display is a stereoscopic display.

In any of the above implementations of the second aspect of the disclosure the first action is to display an indication of the button.

In any of the above implementations of the second aspect of the disclosure the second action is to discontinue display of the indication of the button.

In any of the above implementations of the second aspect of the disclosure the indication of the button is selected from a group of indications consisting of an indication of a function performed upon selection of the button; a change of intensity of an icon displayed on the user interface; and a graphic of the foot of the user relative to a layout of a plurality of buttons, the layout including the button.

In any of the above implementations of the second aspect of the disclosure the first action is to sound a first audible alert and the second action is to sound a second audible alert.

In any of the above implementations of the second aspect of the disclosure the first action is to provide a first haptic feedback and the second action is to provide a second haptic feedback.

In any of the above implementations of the second aspect of the disclosure the first haptic feedback and the second haptic feedback is provided to a hand controller.

A third aspect of the disclosure includes a method of providing feedback upon detection of an object. The method comprises measuring a distance of an object within a field of view of a range sensor. The range sensor positioned with the field of view across a selection surface of a button. The button is positioned for selection by the object. The method comprises performing a first action with a user interface upon the distance measured by the range sensor being less than or equal to a first threshold distance. The method comprises performing a second action with the user interface upon the distance measured by the range sensor being greater than a second threshold distance.

In various implementations of the third aspect of the disclosure, the second threshold distance is greater than the first threshold distance.

In any of the above implementations of the third aspect of the disclosure the first threshold distance is at an edge of the button.

In any of the above implementations of the third aspect of the disclosure the second threshold distance is not coextensive with the button and spaced apart from the edge of the button by a predetermined distance.

In any of the above implementations of the third aspect of the disclosure the predetermined distance is sufficient to mitigate against accidental selection of the button by the object.

In any of the above implementations of the third aspect of the disclosure the predetermined distance is in a range of distances from 10 mm to 30 mm.

In any of the above implementations of the third aspect of the disclosure the object is any one of a non-hand limb, a foot, a leg, a knee, a head, an elbow or extension from human anatomy.

In any of the above implementations of the third aspect of the disclosure the range sensor is any of a time-of-flight distance sensor, a triangulation distance sensor, an optical distance sensor, an acoustic distance sensor, an inductive distance sensor, a capacitive distance sensor, a photoelectric distance sensor, a camera, an infrared distance sensor, a laser range finder, or a light detection and ranging sensor.

In any of the above implementations of the third aspect of the disclosure a direction of the field of view is parallel to a direction of the distance measured by the range sensor.

In any of the above implementations of the third aspect of the disclosure a direction of the field of view is orthogonal to a direction of the distance measured by the range sensor.

In any of the above implementations of the third aspect of the disclosure, the method further comprises measuring a second distance of a second object within a second field of view of a second range sensor. The second range sensor positioned with the second field of view across a second selection surface of a second button. The second button is positioned for selection by the object. The method further comprises performing a third action with the user interface upon the second distance measured by the range sensor being less than or equal to a third threshold distance. The method further comprises performing a fourth action with the user interface upon the second distance measured by the second range sensor being greater than a fourth threshold distance. The fourth threshold distance is greater than the third threshold distance.

In any of the above implementations of the third aspect of the disclosure the third action is different than the first action.

In any of the above implementations of the third aspect of the disclosure the first action is to display an indication of the button.

In any of the above implementations of the third aspect of the disclosure the second action is to discontinue display of the indication of the button.

In any of the above implementations of the third aspect of the disclosure the indication of the button is selected from a group of indications consisting of an indication of a function performed upon selection of the button; a change of intensity of an icon displayed on the user interface; and a graphic of the object relative to a layout of selection buttons, the layout including the button.

In any of the above implementations of the third aspect of the disclosure the first action is to sound a first audible alert and the second action is to sound a second audible alert.

In any of the above implementations of the third aspect of the disclosure the first action is to provide a first haptic feedback and the second action is to provide a second haptic feedback.

In any of the above implementations of the third aspect of the disclosure the first haptic feedback and the second haptic feedback is provided to a hand controller.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1A is a schematic view of a medical system, in accordance with various aspects of the disclosure.

FIG. 1B is a perspective view of an assembly, in accordance with various aspects of the disclosure.

FIG. 1C is a perspective view of a surgeon's control console for a medical system, in accordance with various aspects of the disclosure.

FIG. 2 is a perspective view of a user input tray according to some implementations.

FIG. 3 is a cross-sectional view of the user input tray according to some implementations.

FIGS. 4A-4F illustrate a graphical user interface with icons providing status information about user input devices in the user input tray according to some implementations.

FIGS. 5A, 5B, 5C, and 5D illustrate a graphical user interface with synthetic indicators providing status information about user input devices associated with onscreen tools, according to some implementations.

FIGS. 6A, 6B, 6C, and 6D illustrate a graphical user interface with synthetic indicators providing status information about user input devices associated with onscreen tools, according to some implementations.

FIGS. 7A, 7B, 7C, and 7D illustrate a graphical user interface with synthetic indicators that may conditionally move to stay visible as the components or the endoscope generating the field of view are moved, according to some implementations.

FIG. 8A is a flowchart of operation of a control system according to various implementations described herein.

FIG. 8B is a flowchart of an example threshold-based hysteresis of the control system according to various implementations described herein

FIG. 9 is a flowchart of a calibration operation according to various implementations described herein.

FIG. 10 is a flowchart of a user intent determination according to various implementations described herein.

FIG. 11 illustrates an exemplary computer system.

### DETAILED DESCRIPTION

It should be understood at the outset that although illustrative implementations of one or more implementations are illustrated below, the disclosed systems and methods may be implemented using any number of techniques, whether currently known or in existence. The disclosure should in no way be limited to the illustrative implementations, drawings, and techniques illustrated below, but may be modified within the scope of the appended claims along with their full scope of equivalents. Use of the phrase “and/or” indicates that any one or any combination of a list of options can be used. For example, “A, B, and/or C” means “A”, or “B”, or “C”, or “A and B”, or “A and C”, or “B and C”, or “A and B and C”.

In robot-assisted medical procedures, endoscopic images of the surgical environment may provide a clinician with a field of view of the patient anatomy and any medical tools located in the patient anatomy. Augmenting the endoscopic images with various indicators may allow the clinician to access information while maintaining the field of view. Such indicators may include indicators for components outside of a field of view.

For example, while using an operator input system during surgery, the surgeon places their head against a viewing module to view an endoscopic view of the patient's inner body cavity. It is challenging to operate user input devices with non-hand limbs, such as manipulation of foot pedals in a pedal tray with a foot, without looking at the user input devices. Object presence sensors provide the surgeon with UI indication (e.g., display, audio, or haptic feedback) of which pedals their feet are positioned over, such that the surgeon does not need to look at their feet or use tactile feedback to identify their foot position prior to using a pedal during surgery. This feature is designed to save the surgeon time, reduces the surgeon's task switching and cognitive load, and helps to reduce the likelihood of an accidental pedal press due to inaccurate foot placement during pedal usage.

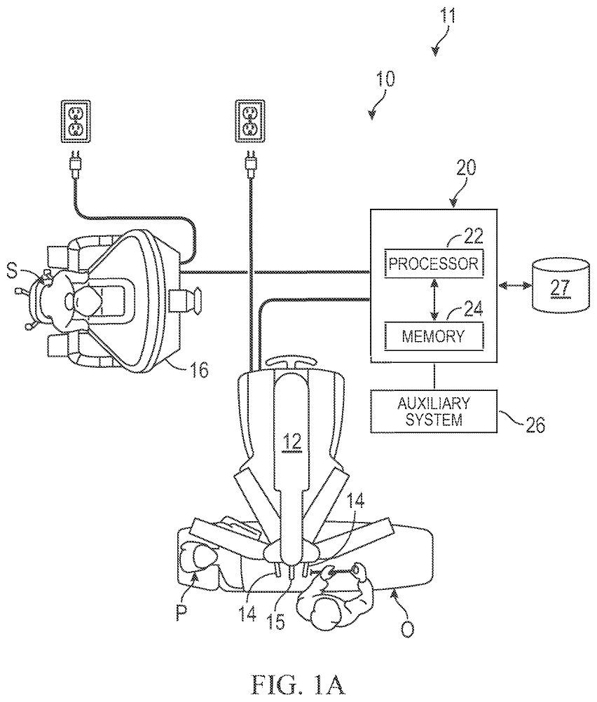

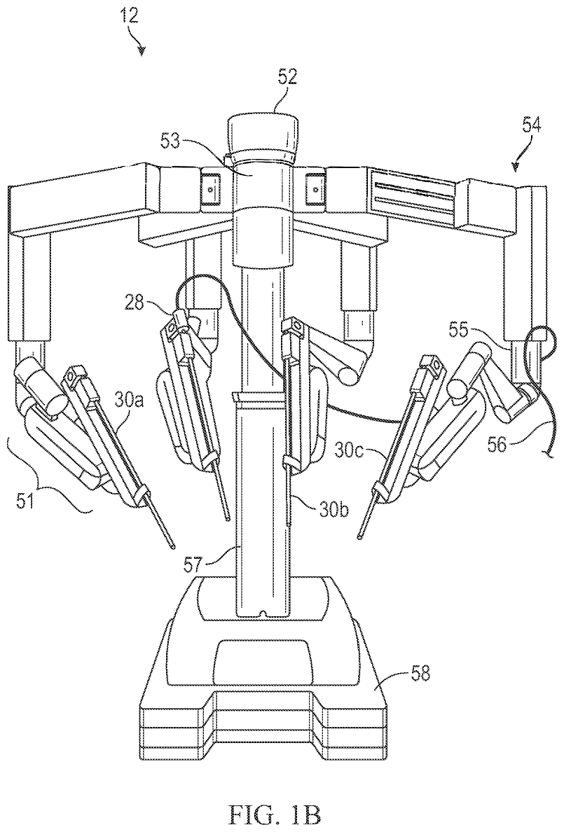

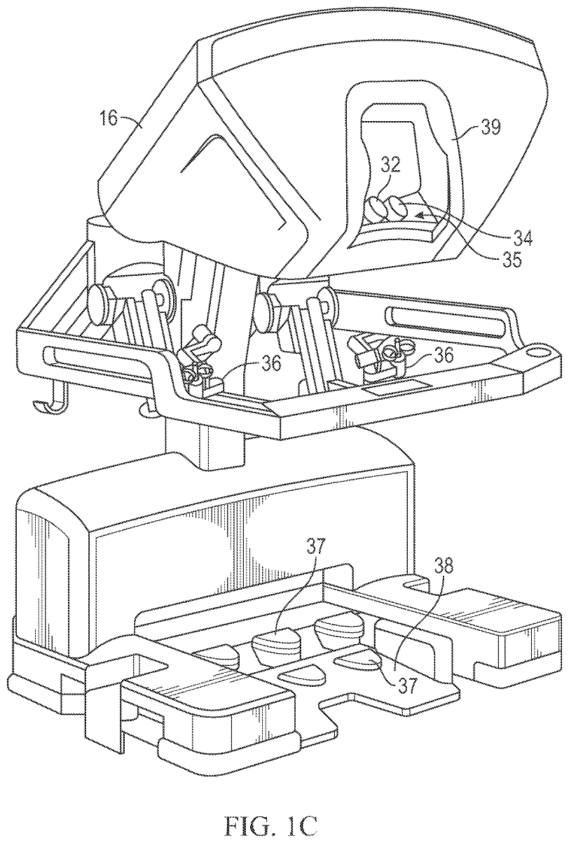

FIGS. 1A, 1B, and 1C together provide an overview of a medical system 10 that may be used in, for example, medical procedures including diagnostic, therapeutic, or surgical procedures. The medical system 10 is located in a medical environment 11. The medical environment 11 is depicted as an operating room in FIG. 1A. In other implementations, the medical environment 11 may be an emergency room, a medical training environment, a medical laboratory, or some other type of environment in which any number of medical procedures or medical training procedures may take place. In still other implementations, the medical environment 11 may include an operating room and a control area located outside of the operating room.

In one or more implementations, the medical system 10 may be a robot-assisted medical system that is under the teleoperational control of a surgeon. In alternative implementations, the medical system 10 may be under the partial control of a computer programmed to perform the medical procedure or sub-procedure. In still other alternative implementations, the medical system 10 may be a fully automated medical system that is under the full control of a computer programmed to perform the medical procedure or sub-procedure with the medical system 10. One example of the medical system 10 that may be used to implement the systems and techniques described in this disclosure is the da Vinci® Surgical System manufactured by Intuitive Surgical, Inc. of Sunnyvale, California.

As shown in FIG. 1A, the medical system 10 generally includes an assembly 12, which may be mounted to or positioned near an operating table O on which a patient P is positioned. The assembly 12 may be referred to as a patient side cart, a surgical cart, or a surgical robot. In one or more implementations, the assembly 12 may be a teleoperational assembly. The teleoperational assembly may be referred to as, for example, a teleoperational arm cart. A medical instrument system 14 and an endoscopic imaging system 15 are operably coupled to the assembly 12. An operator input system 16 allows a surgeon S or other type of clinician to view images of or representing the surgical site and to control the operation of the medical instrument system 14 and/or the endoscopic imaging system 15.

The medical instrument system 14 may comprise one or more medical instruments. In implementations in which the medical instrument system 14 comprises a plurality of medical instruments, the plurality of medical instruments may include multiple of the same medical instrument and/or multiple different medical instruments. Similarly, the endoscopic imaging system 15 may comprise one or more endoscopes. In the case of a plurality of endoscopes, the plurality of endoscopes may include multiple of the same endoscope and/or multiple different endoscopes.

The operator input system 16 may be located at a surgeon's control console, which may be located in the same room as operating table O. In some implementations, the surgeon S and the operator input system 16 may be located in a different room or a completely different building from the patient P. The operator input system 16 generally includes one or more control device(s) for controlling the medical instrument system 14. The control device(s) may include one or more of any number of a variety of input devices, such as hand grips, joysticks, trackballs, data gloves, trigger-guns, foot pedals, hand-operated controllers, voice recognition devices, touch screens, body motion or presence sensors, and other types of input devices.

In some implementations, the control device(s) will be provided with the same degrees of freedom as the medical instrument(s) of the medical instrument system 14 to provide the surgeon with telepresence, which is the perception that the control device(s) are integral with the instruments so that the surgeon has a strong sense of directly controlling instruments as if present at the surgical site. In other implementations, the control device(s) may have more or fewer degrees of freedom than the associated medical instruments and still provide the surgeon with telepresence. In some implementations, the control device(s) are manual input devices that move with six degrees of freedom, and which may also include an actuatable handle for actuating instruments (for example, for closing grasping jaw end effectors, applying an electrical potential to an electrode, delivering a medicinal treatment, and actuating other types of instruments).

The assembly 12 supports and manipulates the medical instrument system 14 while the surgeon S views the surgical site through the operator input system 16. An image of the surgical site may be obtained by the endoscopic imaging system 15, which may be manipulated by the assembly 12. The assembly 12 may comprise endoscopic imaging systems 15 and may similarly comprise multiple medical instrument systems 14 as well. The number of medical instrument systems 14 used at one time will generally depend on the diagnostic or surgical procedure to be performed and on space constraints within the operating room, among other factors. The assembly 12 may include a kinematic structure of one or more non-servo controlled links (e.g., one or more links that may be manually positioned and locked in place, generally referred to as a set-up structure) and a manipulator. When the manipulator takes the form of a teleoperational manipulator, the assembly 12 is a teleoperational assembly. The assembly 12 includes a plurality of motors that drive inputs on the medical instrument system 14. In an implementation, these motors move in response to commands from a control system (e.g., control system 20). The motors include drive systems which when coupled to the medical instrument system 14 may advance a medical instrument into a naturally or surgically created anatomical orifice. Other motorized drive systems may move the distal end of said medical instrument in multiple degrees of freedom, which may include three degrees of linear motion (e.g., linear motion along the X, Y, Z Cartesian axes) and three degrees of rotational motion (e.g., rotation about the X, Y, Z Cartesian axes). Additionally, the motors may be used to actuate an articulable end effector of the medical instrument for grasping tissue in the jaws of a biopsy device or the like. Medical instruments of the medical instrument system 14 may include end effectors having a single working member such as a scalpel, a blunt blade, an optical fiber, or an electrode. Other end effectors may include, for example, forceps, graspers, scissors, or clip appliers.

The medical system 10 also includes a control system 20. The control system 20 includes at least one memory 24 and at least one processor 22 for effecting control between the medical instrument system 14, the operator input system 16, and other auxiliary systems 26 which may include, for example, imaging systems, audio systems, fluid delivery systems, display systems, illumination systems, steering control systems, irrigation systems, and/or suction systems. A clinician may circulate within the medical environment 11 and may access, for example, the assembly 12 during a set up procedure or view a display of the auxiliary system 26 from the patient bedside.

Though depicted in FIG. 1A as being external to other components of medical system 10, the control system 20 may, in some implementations, be contained wholly or partially within any of the assembly 12, operator input system 16, or auxiliary system 26. The control system 20 also includes programmed instructions (e.g., stored on a non-transitory, computer readable medium) to implement some or all of the methods described in accordance with aspects disclosed herein. While the control system 20 is shown as a single block in the simplified schematic of FIG. 1A, the control system 20 may include two or more data processing circuits with one portion of the processing optionally being performed on or adjacent the assembly 12, another portion of the processing being performed at the operator input system 16, and the like.

Any of a wide variety of centralized or distributed data processing architectures may be employed. Similarly, the programmed instructions may be implemented as a number of separate programs or subroutines, or they may be integrated into a number of other aspects of the systems described herein, including teleoperational systems. In one implementation, the control system 20 supports wireless communication protocols such as Bluetooth, IrDA, HomeRF, IEEE 802.11, DECT, and Wireless Telemetry.

The control system 20 is in communication with a database 27 which may store one or more clinician profiles, a list of patients and patient profiles, a list of procedures to be performed on patients, a list of clinicians scheduled to perform procedures, other information, or combinations thereof. A clinician profile may comprise information about a clinician, including how long the clinician has worked in the medical field, the level of education attained by the clinician, the level of experience the clinician has with the medical system 10 (or similar systems), or any combination thereof.

The database 27 may be stored in the memory 24 and may be dynamically updated. Additionally or alternatively, the database 27 may be stored on a device such as a server or a portable storage device that is accessible by the control system 20 via an internal network (e.g., a secured network of a medical facility or a teleoperational system provider) or an external network (e.g., the Internet). The database 27 may be distributed throughout two or more locations. For example, the database 27 may be present on multiple devices which may include the devices of different entities and/or a cloud server. Additionally or alternatively, the database 27 may be stored on a portable user-assigned device such as a computer, a mobile device, a smart phone, a laptop, an electronic badge, a tablet, a pager, and other similar user devices.

In some implementations, control system 20 may include one or more servo controllers that receive force and/or torque feedback from the medical instrument system 14. Responsive to the feedback, the servo controllers transmit signals to the operator input system 16. The servo controller(s) may also transmit signals instructing assembly 12 to move the medical instrument system(s) 14 and/or endoscopic imaging system 15 which extend into an internal surgical site within the patient body via openings in the body. Any suitable conventional or specialized servo controller may be used. A servo controller may be separate from, or integrated with, assembly 12. In some implementations, the servo controller and assembly 12 are provided as part of a teleoperational arm cart positioned adjacent to the patient's body.

The control system 20 can be coupled with the endoscopic imaging system 15 and can include a processor to process captured images for subsequent display, such as to a surgeon on the surgeon's control console, or on another suitable display located locally and/or remotely. For example, where a stereoscopic endoscope is used, the control system 20 can process the captured images to present the surgeon with coordinated stereo images of the surgical site. Such coordination can include alignment between the opposing images and can include adjusting the stereo working distance of the stereoscopic endoscope.

In alternative implementations, the medical system 10 may include more than one assembly 12 and/or more than one operator input system 16. The exact number of assemblies 12 will depend on the surgical procedure and the space constraints within the operating room, among other factors. The operator input systems 16 may be collocated or they may be positioned in separate locations. Multiple operator input systems 16 allow more than one operator to control one or more assemblies 12 in various combinations. The medical system 10 may also be used to train and rehearse medical procedures.

FIG. 1B is a perspective view of one implementation of an assembly 12 which may be referred to as a patient side cart, surgical cart, teleoperational arm cart, or surgical robot. The assembly 12 shown provides for the manipulation of three surgical tools 30a, 30b, and 30c (e.g., medical instrument systems 14) and an imaging device 28 (e.g., endoscopic imaging system 15), such as a stereoscopic endoscope used for the capture of images of the site of the procedure. The imaging device may transmit signals over a cable 56 to the control system 20. Manipulation is provided by teleoperative mechanisms having a number of joints. The imaging device 28 and the surgical tools 30a-c can be positioned and manipulated through incisions in the patient so that a kinematic remote center is maintained at the incision to minimize the size of the incision. Images of the surgical site can include images of the distal ends of the surgical tools 30a-c when they are positioned within the field-of-view of the imaging device 28.

The assembly 12 includes a drivable base 58. The drivable base 58 is connected to a telescoping column 57, which allows for adjustment of the height of arms 54. The arms 54 may include a rotating joint 55 that both rotates and moves up and down. Each of the arms 54 may be connected to an orienting platform 53. The arms 54 may be labeled to facilitate trouble shooting. For example, each of the arms 54 may be emblazoned with a different number, letter, symbol, other identifier, or combinations thereof. The orienting platform 53 may be capable of 360 degrees of rotation. The assembly 12 may also include a telescoping horizontal cantilever 52 for moving the orienting platform 53 in a horizontal direction.

In the present example, each of the arms 54 connects to a manipulator arm 51. The manipulator arms 51 may connect directly to a medical instrument, e.g., one of the surgical tools 30a-c. The manipulator arms 51 may be teleoperable. In some examples, the arms 54 connecting to the orienting platform 53 may not be teleoperable. Rather, such arms 54 may be positioned as desired before the surgeon S begins operation with the teleoperative components. Throughout a surgical procedure, medical instruments may be removed and replaced with other instruments such that instrument to arm associations may change during the procedure.

Endoscopic imaging systems (e.g., endoscopic imaging system 15 and imaging device 28) may be provided in a variety of configurations including rigid or flexible endoscopes. Rigid endoscopes include a rigid tube housing a relay lens system for transmitting an image from a distal end to a proximal end of the endoscope. Flexible endoscopes transmit images using one or more flexible optical fibers. Digital image-based endoscopes have a “chip on the tip” design in which a distal digital sensor such as a one or more charge-coupled device (CCD) or a complementary metal oxide semiconductor (CMOS) device store image data. Endoscopic imaging systems may provide two- or three-dimensional images to the viewer. Two dimensional images may provide limited depth perception. Three-dimensional stereo endoscopic images may provide the viewer with more accurate depth perception. Stereo endoscopic instruments employ stereo cameras to capture stereo images of the patient anatomy. An endoscopic instrument may be a fully sterilizable assembly with the endoscope cable, handle and shaft all rigidly coupled and hermetically sealed.

FIG. 1C is a perspective view of an implementation of the operator input system 16 at the surgeon's control console. The operator input system 16 includes a left eye display 32 and a right eye display 34 for presenting the surgeon S with a coordinated stereo view of the surgical environment that enables depth perception (e.g., left and right eye displays 32, 34 are a stereoscopic display). The left and right eye displays 32, 34 may be components of a display system 35. The left and right eye displays 32, 34 may be incorporated into a head rest 39. A surgeon S may place their head on the head rest 39 for viewing the left and right eye displays 32, 34. In some implementations, the display system 35 may include one or more other types of displays. The display system 35 may present images captured, for example, by the imaging system 15 to display the endoscopic field of view to the surgeon S. The endoscopic field of view may be augmented by virtual or synthetic menus, indicators, and/or other graphical or textual information to provide additional information to the viewer. In some implementations, the display system 35 may include one or more other user feedback devices, such as a lighting system, speaker, haptic feedback device, or other user interface device for conveying information to the surgeon S. In some implementations, the other user interface devices or displays may be positioned apart from the display system 35 on the operator input system 16, the assembly 12, or the control system 20.

The operator input system 16 further includes one or more input control devices 36, which in turn cause the assembly 12 to manipulate one or more instruments of the endoscopic imaging system 15 and/or medical instrument system 14. The input control devices 36 can provide the same degrees of freedom as their associated instruments to provide the surgeon S with telepresence, or the perception that the input control devices 36 are integral with said instruments so that the surgeon has a strong sense of directly controlling the instruments. To this end, position, force, and tactile feedback sensors (not shown) may be employed to transmit position, force, and tactile sensations from the medical instruments, e.g., surgical tools 30a-c, or imaging device 28, back to the surgeon's hands through the input control devices 36. Aspects of the operator input system 16, the assembly 12, and the auxiliary systems 26 may be adjustable and customizable to meet the physical needs, skill level, or preferences of the surgeon S.

Input control devices 37 are foot pedals that receive input from a user's foot. The input control devices 37 are positioned at a base of the operator input system 16 in a user input tray 38. The input control devices 37 may control functions of a teleoperational assembly (e.g., medical system 10, assembly 12) and/or medical tools (e.g., surgical tools 30a-c, or imaging device 28) coupled to the arms 54 of the assembly 12.

While the input control devices 37 are described in the examples presented herein as foot pedals, the input control devices 37 may include any suitable pedal, button, or other user input device for manipulation by a non-hand limb, such as a foot, leg, knee, arm, elbow, head, or other part of human anatomy or extension from human anatomy (e.g., selection wand, cane, or other selection tool) other than a hand that has a comparatively reduced tactile agility and/or sensitivity.

During a medical procedure performed using the medical system 10, the surgeon S or another clinician may need to access medical tools in the patient anatomy that are outside of the field of view of the imaging system 15, may need to engage input control devices 37 (e.g., foot pedals) to activate medical tools or perform other system functions, and/or may need to identify tools that are occluded in the field of view. Further, with a stereoscopic field of view, it may be desirable that synthetic elements presented with the field of view are displayed at depths that correspond with the tissue or components indicated by the synthetic elements. Thus, the synthetic elements may appear to be attached to the components in the field of view rather than floating in front of the field of view. The various implementations described below provide methods and systems that allow the surgeon S to view depth-aware graphical indicators, indicators for components outside of a field of view, and indicators for components occluded in the field of view.

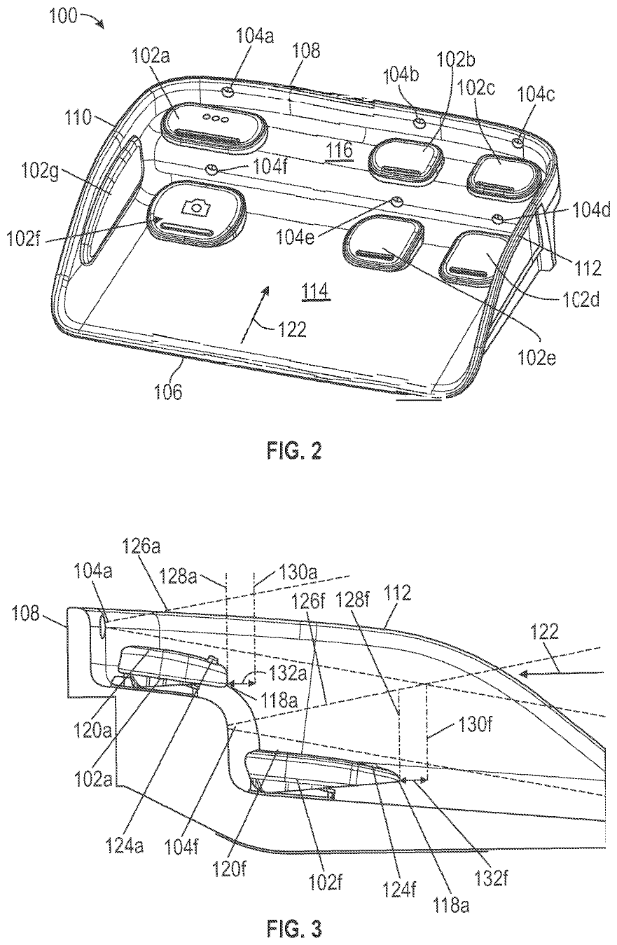

FIG. 2 is a perspective view of a user input tray 100 according to some implementations. The user input tray 100 may be implemented as the user input tray 38, described above. The user input tray 100 includes a plurality of user input devices 102a-102f, singularly or collectively user input device(s) 102. The user input devices 102 are arranged in a layout on one or more surfaces of the user input tray 100. In an implementation, the user input tray 100 is arranged at a base of the operator input system 16 in a layout to facilitate selection of the user input devices 102 by a foot of the surgeon S while looking at the field of view of the imaging system 15 in the display system 35.

The user input tray 100 has a first side 106, also referred to as a front side, and a second side 108, also referred to as a back side, where the second side 108 is opposite from the first side 106. The user input tray 100 also has a third side 110, also referred to as a left side, and a fourth side 112, also referred to as a right side, where the fourth side 112 is opposite to the third side 110. The third side 110 and the fourth side 112 form an angle with the first side 106 and the second side 108 respectively to form a perimeter of the user input tray 100. The user input tray 100 also has a first base 114 and a second base 116. The second base 116 is spaced apart from the first base 114 to form a step within the user input tray 100. A sidewall extends between the first base 114 to the second base 116 to form the step in the user input tray 100. A sidewall extends from the second base 116 along the second side 108. The third side 110, and the fourth side 112 include sidewalls that extend from the first base 114 and the second base 116 to form a partially enclosed area within the user input tray 100. In the example shown, the first side 106 does not include a sidewall. In the example shown, the sidewalls on the third side 110 and the fourth side 112 are tapered towards the first side 106.

FIG. 3 is a cross-sectional view of the user input tray 100 according to some implementations. The user input devices 102 may be referred to as buttons, pedals, touch sensors, or any other device for receipt of user selection input. Each of the user input devices 102 have a leading edge 118 and a selection surface 120. Each of the user input devices 102 is in communication with the control system 20 for registering selection of one or more of the user input devices 102 and performing an associated action to control functions of a teleoperational assembly (e.g., medical system 10, assembly 12) and/or medical tools (e.g., surgical tools 30a-c, or imaging device 28) coupled to the arms 54 of the assembly 12 (e.g., performing one or more functions of the imaging device 28 and/or the surgical tools 30a-c).

The leading edge 118 of the user input devices 102 are positioned to face an anticipated direction of approach 122 of an object (e.g., non-hand limb, such as a foot of surgeon S) when selecting the user input devices 102. For example, when selecting the user input devices 102, an object (e.g., non-hand limb, such as a foot of surgeon S) is anticipated to approach the user input devices 102 from the first side 106 towards the second side 108 of the user input tray 100. In the example shown in FIG. 3, the leading edge 118a of the user input device 102a is positioned closest to the first side 106 of the user input tray 100. Likewise, the leading edge 118f of the user input device 102f is positioned closest to the first side 106 of the user input tray 100. However, a location of the leading edge 118f of the user input device 102f is at a different location than the leading edge 118a of the user input device 102a.

The selection surface 120 of the user input devices 102 is configured to register a selection event. For example, upon an object (e.g., non-hand limb, such as a foot of surgeon S) pressing on or remaining in contact with the selection surface 120 for a predetermined period of time, a selection event is registered on the user input device 102. As noted above, upon registration of a selection event on one of the user input devices 102, the control system 20 controls an associated function(s) of the medical system 10, the assembly 12, the arms 54 of the assembly 12, surgical tools 30a-c, and/or imaging device 28. In various examples, the user input devices 102 include one or more sensors (not shown) for registering a selection event. For example, the one or more sensors may include a pressure sensors, tactile sensor, displacement sensors, switch, button, capacitive sensor, or other types of sensors that detect that one or more of the user input devices has been activated or engaged.

In various implementations, the one or more sensors in the user input devices 102 for registering a selection event can differentiate between a hover event and a selection event. That is, the one or more sensors in the user input devices 102, alone or together with the range sensors 104, differentiate between an object positioned for selection of the selection surface 120, is resting on the selection surface 120, or in contact with the selection surface 120 (e.g., a hover event) and the object pressing on the selection surface 120 (e.g., a selection event). For example, a hover event may be detected upon the surgeon S moving their foot from one of the user input devices 102 to another.

For example, the one or more sensors may use a combination of first sensor (e.g., a pressure sensor or capacitive sensor) to detect contact with the selection surface 120 and a second sensor (e.g., tactile sensor, displacement sensor, switch, or button) to detect selection of the selection surface 120.

In another example, the one or more sensors may use the same sensor for detecting both contact with the selection surface 120 and selection of the selection surface 120. For example, a first sensor may detect contact with the selection surface 120 (e.g., a pressure sensor detects a first threshold amount of pressure, a multi-stage switch detects a first stage of the multi-stage switch, etc.) and the first sensor may also detect selection of the selection surface 120 (e.g., a pressure sensor detects a second threshold amount of pressure that is greater than the first threshold pressure, a multi-stage switch detects a second stage of the multi-stage switch, etc.).

In various implementations, the selection surface 120 may include a protrusion 124. The protrusion 124 is positioned on the selection surface 120 along the leading edge 118. For example, the protrusion 124a is positioned on the selection surface 120 a along the leading edge 118a. The protrusion 124 provides tactile feedback to aid with positioning an object for selection on the selection surface of the user input devices 102. The protrusion 124 also facilitates grip onto the selection surface 120 of the user input devices 102. For example, the protrusion 124 increases frictional engagement with a shoe or other selection object to aid in positive selection of the user input devices 102. While each of the user input devices 102 are depicted with a protrusion 124, in some implementations only one or a some of the user input devices 102 have a protrusion 124.

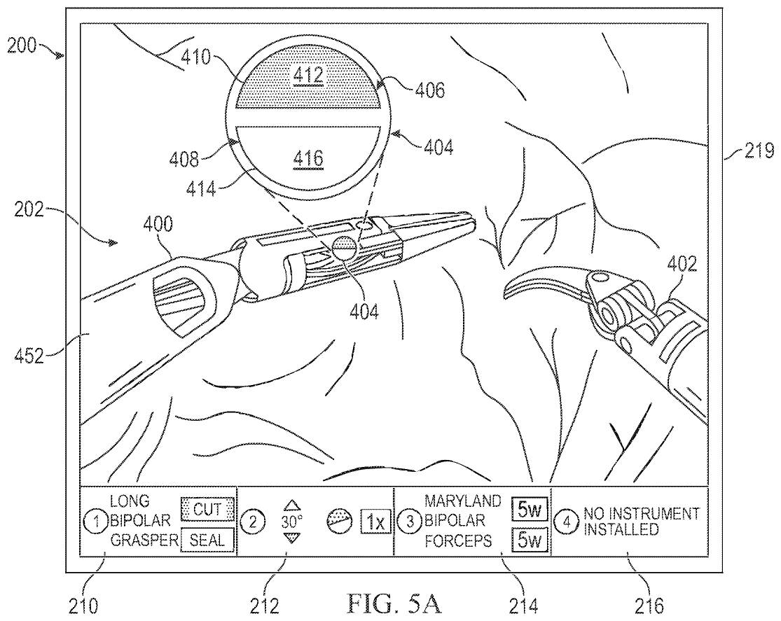

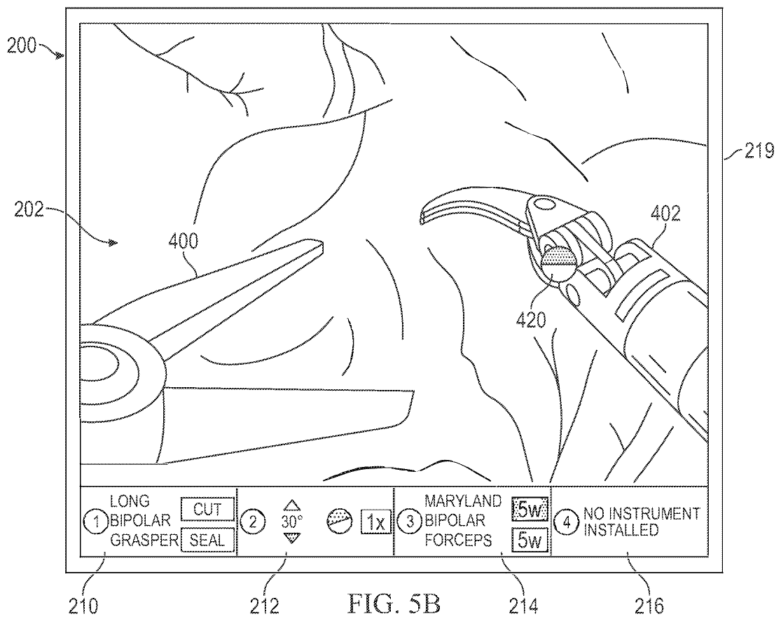

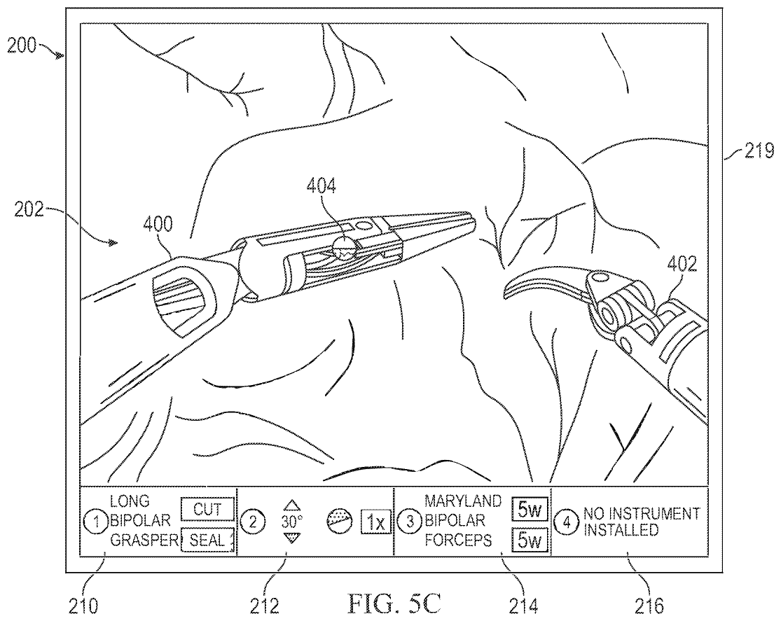

Upon detection of a hover event or a selection event for one or more of the user input devices 102, the control system 20 controls functions of a teleoperational assembly (e.g., medical system 10, assembly 12) and/or medical tools (e.g., surgical tools 30a-c, or imaging device 28) coupled to the arms 54 of the assembly 12. For example, upon detection of a selection event for the user input device 102f, the control system 20 may control the imaging system 15 to capture an image of a current field of view. Controls of other tools and functions performed by the control system 20 are contemplated by this disclosure, such as the tools and functions described in conjunction with FIGS. 5A-7D below.

With reference to FIGS. 2 & 3, a plurality of range sensors 104a-104f, collectively range sensors 104, are positioned with a field of view 126 across the selection surface 120 of the user input devices 102. The range sensors 104 are configured to measure a distance of an object within the field of view 126. Each of the range sensors 104 is in communication with the control system 20 for controlling the display system 35 and/or one or more other user feedback devices, such as a lighting system, speaker, haptic feedback device, or other user interface device for conveying information to the surgeon S.

The range sensors 104 may be any type of sensor for measuring a distance of an object relative to the selection surface 120. For example, the range sensor may be a time-of-flight distance sensor, a triangulation distance sensor, an optical distance sensor, an acoustic distance sensor, an ultrasonic wave sensor, an inductive distance sensor, a capacitive distance sensor, a photoelectric distance sensor, a camera, an infrared distance sensor, a laser range finder, a light detection and ranging sensor, or any other type of range sensor.

In the example shown, one of the range sensors 104 is positioned with a field of view 126 across the selection surface 120 of each of the user input devices 102. For example, the range sensor 104a is positioned on the sidewall that extends from the second base 116 along the second side 108 and faces toward the first side 106 so that the field of view 126a is across the selection surface 120a of the user input device 102a.

In the example shown, the range sensors 104 are positioned to face the anticipated direction of approach 122 of an object. Therefore, a direction of the field of view 126 is parallel to a direction of the distance measured by the range sensor 104. That is, the field of view 126 extends across the selection surface 120 towards the leading edge 118 of the user input devices 102. In other words, the range sensors 104 are positioned to face in a direction from the second side 108 towards the first side 106 of the user input tray 100.

In some implementations, one or more of the range sensors 104 may be positioned to face orthogonal to the anticipated direction of approach 122 of an object. In other words, one or more of the range sensors may be positioned with the field of view 126 that extends in a direction parallel to the leading edge 118 of the user input devices 102. For example, as opposed to being positioned along a sidewall on the second side 108 of the user input tray 100, one or more range sensors 104 may be positioned on a sidewall on the third or fourth side 110, 112 of the user input tray 100.

In an example, a camera may be positioned along the sidewall on the second side 108 of the user input tray 100 to capture an image with a field of view similar to that shown in FIG. 3. Therefore, a direction of the field of view is orthogonal to a direction of the distance measured by the range sensor. The control system 20 may perform image processing of the image to determine a distance of an object relative to the selection surface 120. The camera may be used in combination with a range sensor or in combination with a second camera to generate depth information indicative of where the object may be along the length of the first or second side 106, 108 of the user input tray 100 (e.g., in front of user input device 102a, in front of user input device 102b, or in front of user input device 102c).

Other placements and orientations of the range sensors 104 are contemplated by this disclosure. For example, the range sensors 104 may be placed on the user input devices 102 themselves with a field of view pointing away from the selection surface 120. The range sensors 104 may also be placed above the user input tray 100 with a field of view downward towards the user input devices 102. The range sensors 104 may also be placed on a sidewall on the third or fourth side 110, 112 of the user input tray 100 with a field of view across the user input tray 100.

The user input device 102g is positioned on a sidewall on the third side 110 of the user input tray 100. Therefore, range data from the range sensor 104a and/or the range sensor 104f may be used for detecting an object in proximity to the selection surface 120 of the user input device 102g. More generally, one or more of the range sensors 104 may be positioned with a field of view 126 that extends across the selection surface 120 of a plurality of the user input devices 102.

While only one range sensor 104 is shown to be positioned with a field of view 126 across the selection surface 120 of the user input devices 102, in some implementations, a plurality of range sensors (e.g., two or more) may be used to increase redundancy of object detection and/or increase the field of view 126 across the selection surface 120 of the user input devices 102.

In operation, the range sensors 104 are configured to measure a distance of an object relative to the selection surface 120. At each of a plurality of threshold distance values a different user interface action is performed by the control system 20. The different user interface actions provide feedback (e.g., tactile, visual, auditory) to the surgeon S regarding placement of an object (e.g., non-hand limb, such as a foot of surgeon S) with respect to the user input devices 102 and their activation.

In the example shown in FIG. 3, there are two threshold distances associated with each of the range sensors 104. For example, for the range sensor 104a, a first threshold distance 128a and a second threshold distance 130a, where the second threshold distance 130a is greater than the first threshold distance 128a.

Likewise, the range sensor 104f, a first threshold distance 128f and a second threshold distance 130f, where the second threshold distance 130f is greater than the first threshold distance 128f. In various implementations, the first threshold distance 128a is the same as or different than the first threshold distance 128f. The second threshold distance 130a is the same as or different than the second threshold distance 130f.

More generally, the first threshold distances 128a, 128f are referred to singularly or collectively as a first threshold distance(s) 128 and the second threshold distances 130a, 130f are referred to singularly or collectively as a second threshold distance(s) 130, where the second threshold distance 130 is greater than the first threshold distance 128.

The first threshold distance 128 is positioned at or near the leading edge 118 of the user input device 102. The first threshold distance 128 being positioned near the leading edge 118 of the user input device 102 is a location whereby purposeful or incidental selection of the user input device 102 is likely to occur. The second threshold distance 130 is not coextensive with the user input device 102 (e.g., is not positioned along the selection surface 120) and spaced apart from the leading edge 118 of the user input device 102 by a predetermined distance. In the example shown in FIG. 3, there is a predetermined distance 132a between the first threshold distance 128a and the second threshold distance 130a. Likewise, there is a predetermined distance 132f between the first threshold distance 128f and the second threshold distance 130f. More generally, the predetermined distance 132a, 132f are referred to singularly or collectively as a predetermined distance(s) 132.

In various implementations, a placement of the first and second threshold distance 128, 130 may vary depending on other presences sensors on the medical system 10, the assembly 12, and/or the operator input system 16. For example, hand presence sensors on the input control devices 36 or head presence sensors on the display system 35 and/or the head rest 39 can be used to adjust the first and second threshold distance 128, 130 for detection of an object. If a head and/or hand is not detected, then the first and second threshold distance 128, 130 may be adjusted to make it harder to detect an object (e.g., require more certain measurement of presence of an object before providing an indication of such).

In various implementations, the predetermined distance 132 is between 5 and 50 mm, inclusive of the endpoints. In some implementations, the predetermined distance 132 is between 10 and 30 mm, inclusive of the endpoints. In some implementations, the predetermined distance 132 is between 15 and 25 mm, inclusive of the endpoints. In an implementation, the predetermined distance 132 is 20 mm. All values provided are contemplated to have a variation of up to 25% of the values provided.

In various implementations, the predetermined distance 132 is the same for all of the user input devices 102. In some implementations, the predetermined distance 132 is different for one or more of the user input devices 102 depending on a geometry of the user input devices 102. More generally, the predetermined distance 132 is set to be a sufficient distance away from the user input devices 102 to mitigate against accidental selection of the user input device 102 by the object.

As discussed above, the control system 20 determines a distance measured by one or more of the range sensors 104 to an object (e.g., non-hand limb, such as a foot of surgeon S) within the field of view 126. Upon the control system 20 determining that the measured distance is less than or equal to the first threshold distance 128, the control system 20 performs a first user interface action. Upon the control system 20 determining that the measured distance is greater than or equal to the second threshold distance 130, the control system 20 performs a second user interface action. For example, for range sensor 104f, upon the control system 20 determining that the measured distance is less than or equal to the first threshold distance 128f, the control system 20 performs a first user interaction. Likewise, upon the control system 20 determining that the measure distance for the range sensor 104f is greater than or equal to the second threshold distance 130f, the control system 20 performs a second user interaction.

The first and second user interface actions provide feedback (e.g., tactile, visual, auditory) to the surgeon S regarding placement of an object (e.g., non-hand limb, such as a foot of surgeon S) with respect to the user input devices 102. The first user interface action provides feedback that an object is over or otherwise positioned to facilitate selection of one of the user input devices 102. The second user interface action provides feedback that the object is no longer over or otherwise positioned to facilitate selection of the one of the user input devices 102. In various implementations, the first user interface action may be the same or different for different ones of the user input devices 102. Likewise, the second user interface action may be the same or different for different ones of the user input devices 102.

Following the example associated with the range sensor 104f above, the first user interface action provides feedback that an object is within the field of view 126f and positioned at or closer than the first threshold distance 128f so as to be over or otherwise positioned to facilitate selection of the selection surface 120f of the user input device 102f. The second user interface action provides feedback that the object is at or farther away than the second threshold distance 130f so as to no longer over or otherwise positioned to facilitate selection of the selection surface 120f of the user input device 102f.

In various implementations, the control system 20 registers hover and selection events of the user input devices 102 depending on an order that an object is detected to be within the first threshold distance 128. For example, upon first detecting an object within the first threshold distance 128a, the control system 20 may ignore subsequent detection of an object within the first threshold distance 128f or hover or selection events of the user input device 128e until the object first detected within the first threshold distance 128a is detected to be at or farther than the second threshold distance 130a.

In various implementations, if the control system determines an object is within the first threshold distance 128 for more than one of the user input devices 102, the control system 20 may determine the object is located at the user input device 102 with a closer range reading (e.g., higher signal intensity) from the range sensors 104. For example, a first range reading is provided from the range sensor 104a, and a second range reading is provided from the range sensor 104f, and both are within their respective first threshold distances 128a, 128f. The control system 20 may determine that an object is located at the user input device 102a if the first range reading is closer (e.g., has a higher signal intensity) than the second range reading. In such implementations, the first user interface action is provided for the user input device 102a, but not for the user input device 102f.

Alternatively, the first user interface action may be provided for all user input devices 102 where an object is detected within the first threshold distance. Alternatively, the control system 20 may issue an error or warning upon detection of an object within the threshold distance 128 of more than one of the user input devices 102.

In various implementations, the control system 20 registers hover and selection events of the user input devices 102 depending on whether an object is detected to be within the first threshold distance 128. For example, upon detection of a hover or selection event for user input device 102a, if an object is not detected to be within the first threshold distance 128a, the control system 20 ignores the hover or selection event. Therefore, the range sensors 104 provide redundancy to prevent incidental or unintended selection of one or more of the user input devices 102.

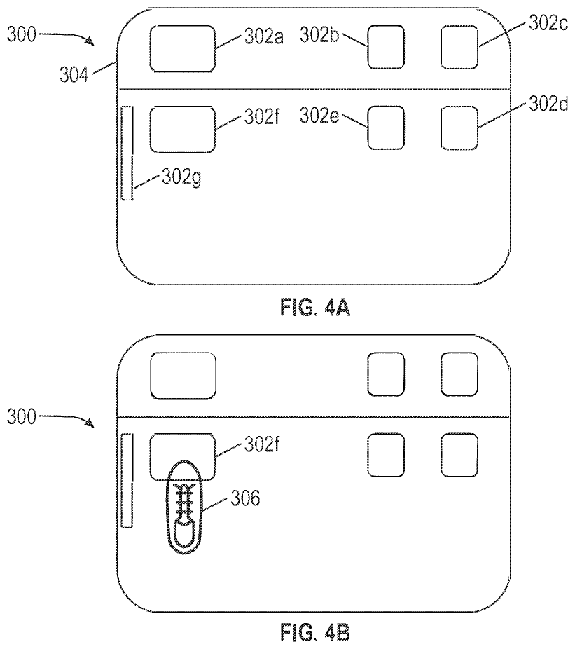

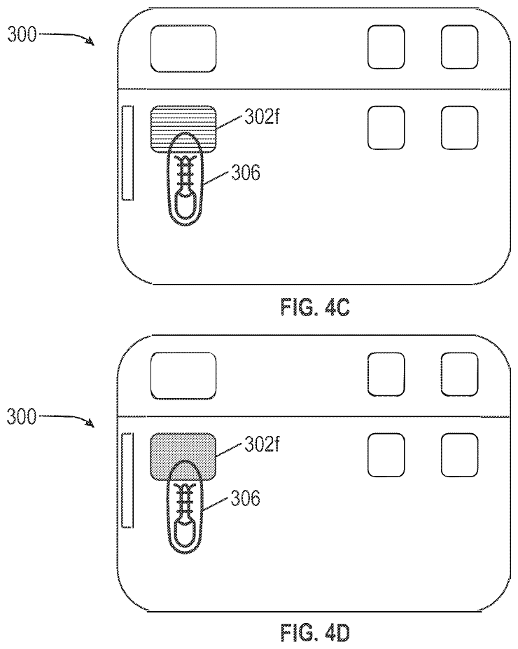

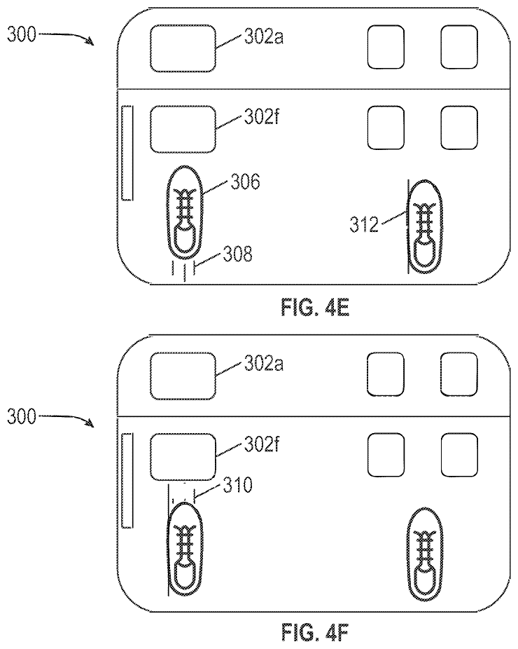

FIGS. 4A-4F illustrate a graphical user interface 300 that may be displayed, for example, on display system 35. The graphical user interface 300 includes icons 302a-302g, individually or collectively icon(s) 302, that correspond to the user input devices 102a-102f in the user input tray 100. The icons 302 provide visual feedback on the display system 35 for the first and second user interface actions. In the examples shown, the icons 302 are arranged in a layout of the user input devices 102 in the foot tray 100. In some implementations, the icons 302 may be arranged in any layout or only be displayed when performing the first or second user interface actions. In some implementations, the graphical user interface 300 also includes an icon 304 indicative of the shape of the user input tray 100.

In some implementations, the graphical user interface 300 may be displayed within a portion of a larger graphical user interface (not shown). For example, as shown and described in FIGS. 5A-7D, the larger graphical user interface may include a field of view portion for displaying an image of a field of view of a surgical environment captured by an imaging system (e.g., imaging system 15). The larger graphical user interface may also include information blocks for displaying information about medical tools and an information block for displaying information about the imaging system capturing the image in the field of view portion. The graphical user interface 300 may be included as a further information block within the larger graphical user interface or overlayed on the field of view portion of the larger graphical user interface.

In some implementations, the first user interface action is to modify the display of an associated one of the icons 302 to indicate that an object (e.g., non-hand limb, such as a foot of surgeon S) is over or otherwise positioned to facilitate selection of one of the user input devices 102. In other words, the first user interface action is to the first action is to display an indication of the user input device 102 where an object is positioned for selection.

In some implementations, the second user interface action is to modify the display of an associated one of the icons 302 to indicate that an object (e.g., non-hand limb, such as a foot of surgeon S) is no longer over or otherwise positioned to facilitate selection of one of the user input devices 102. In various implementations, the second user interface action is to simply discontinue display of the first user interface action.

In an example shown in FIG. 4B, the indication of the user input device 102 where an object is positioned for selection is displayed as a foot icon 306 in an overlapping manner with the icon 302f to indicate that an object is positioned over or otherwise positioned to facilitate selection of the user input device 102f. Therefore, the foot icon 306 serves as a graphic of the object (e.g., non-hand limb, such as a foot of surgeon S) relative to a layout of the icons 302.

In other implementations, the indication of the user input device 102 where an object is positioned for selection is displayed as an indication of a function performed upon selection of a user input device 102. For example, for user input device 102f, an icon of a camera may be displayed in icon 302f or otherwise displayed in the larger graphical user interface described above to indicate that an object is positioned to facilitate activation of a camera function upon selection of the user input device 102f.

In still further implementations, the indication of the user input device 102 where an object is positioned for selection is displayed as a change in intensity, color, highlighting, or other visually distinctive change to the icon 302. For example, as shown in FIG. 4C, the icon 302f is shown displayed with a pattern. In this example, the icon 302f is also shown with the foot icon 306, though in other examples, the foot icon 306 may be omitted.

In another implementation, the graphical user interface 300 may be modified based on sensor readings from both the range sensors 104 and the one or more sensors of the user input devices 102. For example, the graphical user interface 300 may first display the indication of the user input device 102 where an object is positioned for selection. Additionally, upon detection of the object resting on or in contact with the selection surface 120 (e.g., a hover event), the icon 302 may be further modified to indicate the hover event. For example, the indication of the user input device 102 where an object is positioned for selection may be displayed as shown in FIG. 4B. Subsequently, upon detection of the hover event on the user input device 102f, the icon 302f is subsequently modified to show the pattern as shown in FIG. 4C or otherwise modified with a distinctive visual appearance. Additionally or alternatively, upon detection of the object pressing on the selection surface 120 (e.g., a selection event), the icon 302 may be further modified to indicate the selection event. For example, upon detection of the selection event on the user input device 102f, the icon 302f is subsequently modified to show the fill pattern as shown in FIG. 4D or otherwise modified with a distinctive visual appearance.

In various implementations, one or more of the above examples may be used in combination with each other as the indication of the user input device 102 where an object is positioned for selection, as an indication of a hover event, and/or as an indication of a selection event.

In some implementations, the control system 20 tracks and evaluates range data from the range sensors 104 as a time series. Therefore, the control system 20 is additionally able to determine a direction of movement and/or velocity of an object, even outside of the threshold distances discussed above. Such a time series of range data may facilitate determination of an intent of the surgeon S based on the speed and/or direction of motion. Based on the determined intent, the control system 20 may modify user interface actions or operations performed by control system 20.

In some implementations, the control system 20 determines a three dimensional trajectory of an object based on the time series of data using range data from one or more of the range sensors 104. For example, range sensors 104 may capture three dimensional range data. Alternatively or additionally, the control system 20 may integrate range data from more than one of the range sensors 104 at different locations to resolve a three dimensional position, direction of movement, and/or velocity of an object.

For example, upon tracking a time series of range data, the control system 20 may determine that the surgeon S is rapidly (e.g., having a velocity greater than a first predetermined threshold) moving their foot and/or moving their foot in a direction away from the user input devices 102. Therefore, the control system 20 may determine that the surgeon S intends to no longer use the user input devices 102. Accordingly, any incidental hover events or selection events on any of the user input devices 102 may be ignored by the control system 20 or otherwise require verification from the surgeon S.

In another example, upon tracking a time series of range data, the control system 20 may determine that the surgeon S is slowly (e.g., having a velocity less than a second predetermined threshold) moving their foot and/or moving their foot in a direction towards one or more of the user input devices 102. Therefore, the control system 20 may determine that the surgeon S intends to select a user input device 102 in the direction detected.

Upon determining an intent to select one of the user input device 102, the control system 20 may initiate one or more control actions associated with the user input devices 102 in the detected direction that may need a lead time to execute in order reduce a lag time between selection of the user input device and execution of the control action. Alternatively or additionally, the control system 20 may change a power state of a medical tool associated with the user input devices 102 in the detected direction such that the tool may transition from a low power consumption mode to a higher power consumption mode. Alternatively or additionally, the control system 20 may provide user interface feedback to notify the surgeon S of which of the user input devices 102 their foot is currently moving towards. The second predetermined velocity threshold is the same as or different than the first predetermined velocity threshold. Other intents and actions are contemplated by this disclosure.

In another example, upon tracking a time series of range data, the control system 20 may modulate a time period in which a user input device 102 can be selected. For example, upon tracking a time series of range data, the control system 20 may determine that the surgeon S is rapidly moving their foot (e.g., having a velocity greater than a predetermined threshold) and any detected selection events may be ignored or otherwise require verification from surgeon S within a predetermined time period.

Additionally or alternatively, upon tracking a time series of range data, the control system 20 may animate or otherwise modify the graphical user interface 300 to provide an indication of the speed and direction of the object relative to the user input devices 102. As shown in the examples of FIGS. 4E and 4F, the graphical user interface 300 may animate a location of the object even outside of the threshold distances. In some implementations, the graphical user interface 300 may animate multiple objects positioned within the icon 304 indicative of the shape of the user input tray 100. For example, the graphical user interface 300 may animate both left and right feet of the surgeon S as indicated by the foot icon 306 and the foot icon 312.

As shown in FIG. 4E, the foot icon 306 may be animated to move in a direction towards icons 302a and 302f along with an indicator 308 that is representative of a direction and/or velocity of motion of the object. For example, by being positioned on the back side of the foot icon 306, the indicator 308 represents motion of the object towards the user input devices 102. The velocity may be represented with the indicator 308 by having longer lines indicate a higher velocity and shorter lines indicate a slower velocity. Other visual representations of direction and velocity are contemplated by this disclosure.

Likewise, as shown in FIG. 4F the foot icon 306 may be animated to move in a direction away from icons 302a and 302f along with an indicator 310 that is representative of a direction and/or velocity of motion of the object. For example, by being positioned on the front side of the foot icon 306, the indicator 310 represents motion of the object away from the user input devices 102. The velocity may be represented with the indicator 310 by having longer lines indicate a higher velocity and shorter lines indicate a slower velocity. Other visual representations of direction and velocity are contemplated by this disclosure.

In various implementations, the control system 20 may animate graphical user interface 300 with the indication of the object (e.g., the foot icon 306) linearly, two dimensionally, or three dimensionally depending on the sensitivity and resolution of the range sensors 104. For a linear animation, the foot icon 306 may simply travel back and forth in a line that intersects a plurality of the icons 302. For example, as shown in FIG. 4E, the foot icon 306 may animate movement of a left foot of the surgeon S in a line that intersects with the icons 302a and 302f. For a two dimensional animation, the foot icon 306 may be animated to be positioned in any corresponding position of the object within the user input tray 100 (e.g., anywhere within the icon 304 indicative of the shape of the user input tray 100).

In the examples described above with reference to FIGS. 4A-4F, the first and second user interface actions are to modify a display on the display system 35. In other examples, the first and second user interface actions may be to provide auditory or haptic feedback to the surgeon S.

For example, for auditory feedback a first audio indication (e.g., tone, sound effect, music, etc.) may be output from a speaker as the first user interface action. A second audio indication may be output from the speaker as the second user interface action. The first audio indication may be the same or different than the second audio indication. Moreover, different ones of the user input devices 102 may have different sets of first and second audio indications.

For example, a first audio indication may be provided as the first user interface action associated with the user input device 102a, a second audio indication may be provided as the second user interface action associated with the user input device 102a. Likewise, a third audio indication may be provided as the first user interface action associated with the user input device 102f, a fourth audio indication may be provided as the second user interface action associated with the user input device 102f. While only two of the user input devices 102 are discussed in this example, any of the user input devices 102 may have the same or different audio indications for the first and second user interface actions.

Likewise, for haptic feedback different patterns of feedback (e.g., pulse, sequence, etc.) may be provided to the surgeon S. Haptic feedback may be provided to the surgeon S via a haptic feedback transducer (not shown) coupled to any of the user input devices, via a haptic feedback transducer (not shown) coupled to head rest 39, via haptic feedback provided by one or more of the input control devices 36 (e.g., hand controllers), or via haptic feedback provided anywhere on the operator input system 16.

For example, a first haptic feedback pattern may be provided to the surgeon S as the first user interface action. A second haptic feedback pattern may be provided to the surgeon S as the second user interface action. The first haptic feedback pattern may be the same or different than the second haptic feedback pattern. Moreover, different ones of the user input devices 102 may have different sets of first and second haptic feedback patterns.

For example, a first haptic feedback pattern may be provided as the first user interface action associated with the user input device 102a, a second haptic feedback pattern may be provided as the second user interface action associated with the user input device 102a. Likewise, a third haptic feedback pattern may be provided as the first user interface action associated with the user input device 102f, a fourth haptic feedback pattern may be provided as the second user interface action associated with the user input device 102f. While only two of the user input devices 102 are discussed in this example, any of the user input devices 102 may have the same or different haptic feedback patterns for the first and second user interface actions.

FIGS. 5A, 5B, 5C, and 5D illustrate a graphical user interface 200 that may be displayed, for example, on display system 35. The graphical user interface 200 may include a field of view portion 202 for displaying an image of a field of view of a surgical environment captured by an imaging system (e.g., imaging system 15). The surgical environment may have a Cartesian coordinate system Xs, Y s, Zs. The image in the field of view portion 202 may be a three-dimensional, stereoscopic image and may include patient tissue and surgical components including instruments such as a medical tool 400 and a medical tool 402. The graphical user interface 200 may also include an information block 210 displaying information about medical tool 400, an information block 212 displaying information about the imaging system (e.g., imaging system 15) capturing the image in the field of view portion 202, an information block 214 displaying information about the medical tool 402, and an information block 216 displaying information indicating a fourth medical tool is not installed. The information blocks 210, 212, 214, 216 may include the tool type, the number of the manipulator arm to which the tool is coupled, status information for the arm or the tool, and/or operational information for the arm or the tool.

The medical tool 400 and the medical tool 402 are visible in the field of view portion 202. Functions of the medical tools may be initiated by engaging corresponding user input devices 102 (e.g., foot pedals) on the user input tray 100. For example, the medical tool 400 may be operated by manipulator arm 1 as indicated in information block 210 and may be a vessel sealer that may perform the function of cutting when the user input device 102b is engaged and may perform the function of sealing when the user input device 102e is engaged. As shown in FIG. 5A, the tool 400 may be labeled with a synthetic indicator 404. In this implementation, the synthetic indicator 404 may be a generally circular badge including an upper semi-circular portion 406 and a lower semi-circular portion 408. The upper semi-circular portion 406 includes an outline portion 410 and a central portion 412, and the lower semi-circular portion 408 includes an outline portion 414 and a central portion 416. The upper semi-circular portion 406 may correspond to the function of the secondary user input device 102b and may indicate the engagement status (e.g., hovered, activated) of the user input device 102b. The lower semi-circular portion 408 may correspond to the function of the primary user input device 102e and may indicate the engagement status (e.g., hovered, activated) of the user input device 102e. The spatial relationship of the upper semi-circular portion 406 and the lower semi-circular portion 408 may have the same or a similar spatial relationship as the user input devices 102b, 102e. When the range sensors 104 and/or sensors of the user input devices 102 detect that an operator's foot is hovering above or otherwise within a threshold distance from the user input device 102b, the outline portion 410 of the upper semi-circular portion 406 may change appearance (e.g., change color, become animated) to indicate to the operator that the operator's foot is near the user input device 102b. Thus, the operator can determine the foot position while the operator's vision remains directed to the graphical user interface 200. When the operator engages the user input device 102b (e.g., steps on or depresses the pedal), the central portion 412 of the upper semi-circular portion 406 may change appearance (e.g., change color, become animated) to indicate to the operator that the operator's foot has engaged the user input device 102b and the function of the user input device 102b (e.g., cutting) has been initiated. In some implementations, the hover or engaged status of the user input device 102b may be indicated in the information block 210 using the same or similar graphical indicators. The left bank of user input devices 102 (e.g., user input devices 102b, 102e) may be associated with left hand input control devices, and the right bank of user input devices 102 (e.g., user input devices 102c, 102d) may be associated with right hand input control devices. Each hand may be associated to control any instrument arm. The co-located synthetic indicators reflect this association of an instrument to a corresponding hand & foot. In some configurations, the instrument pose with respect to the endoscopic field of view may otherwise appear to have an ambiguous association to a left or right side, so the co-located synthetic indicator clarifies this association.

As shown in FIG. 5C, the lower semi-circular portion 408 may function, similarly to the upper semi-circular portion 406, as an indicator for the hover and engagement of the user input device 102e. When the operator engages the primary user input device 102e (e.g., steps on or depresses the pedal), the central portion of the lower semi-circular portion 408 may change appearance (e.g., change color, become animated) to indicate to the operator that the operator's foot has engaged the user input device 102e and the function of the user input device 102e (e.g., sealing) has been initiated. The user input devices 102 at the surgeon's console may be color-coded. For example, primary user input devices 102e, 102d may be colored blue and the secondary user input devices 102b, 102c may be colored yellow. This color-coding is reflected in the associated highlight and fill colors of the pedal function synthetic indicators on the graphical user interface.

As shown in FIG. 5B, the tool 402 may be labeled with a synthetic indicator 420. In this implementation, the synthetic indicator 420 may be substantially similar in appearance and function to the synthetic indicator 404 but may provide information about the set of user input device 102c, 102d. The tool 402 may be operated by manipulator arm 3 as indicated in information block 214 and may be a monopolar cautery instrument that may perform the function of delivering an energy for cutting when the user input device 102c is engaged and may perform the function of delivering an energy for coagulation when the user input device 102d is engaged. When the range sensors 104 and/or sensors of the user input devices 102 detect that an operator's foot is hovering above or otherwise within a threshold distance from the secondary user input device 102c, an outline portion of an upper semicircular portion may change appearance to indicate to the operator that the operator's foot is near the user input device 102c. When the range sensors 104 and/or sensors of the user input devices 102 determine that the operator has engaged or activated the user input device 102c, a central portion of the upper semi-circular portion may change appearance to indicate to the operator that the operator's foot has engaged the user input device 102c and the function of the user input device 102c (e.g., delivering energy for cutting) has been initiated. In some implementations, the hover or engaged status of the secondary user input device 102b may be indicated in the information block 214 using the same or similar graphical indicators.

As shown in FIG. 5D, the lower semi-circular portion of indicator 420 may function, similarly to the upper semi-circular portion, as an indicator for the hover and engagement of the primary user input device 102d. When the operator engages the primary user input device 102d, the central portion of the lower semi-circular portion may change appearance to indicate to the operator that the operator's foot has engaged the primary user input device 102d and the function of the user input device 102d (e.g., delivering energy for coagulation) has been initiated.

The position and orientation of synthetic indicators 404, 420 may be determined to create the appearance that the synthetic indicators are decals adhered, for example, to the tool clevis or shaft. As the tools or endoscope providing the field of view are moved, the synthetic indicators 404, 420 may change orientation in three-dimensional space to maintain tangency to the tool surface and to preserve the spatial understanding of upper and lower pedals.

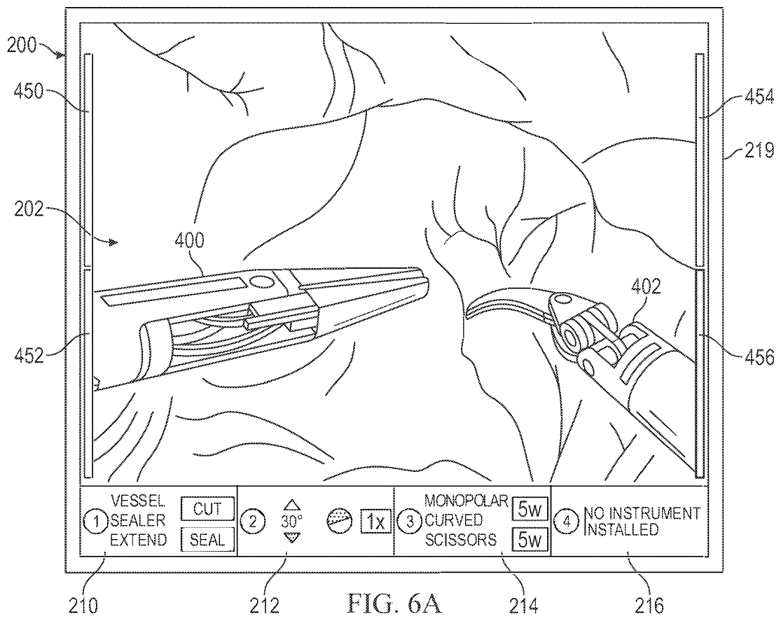

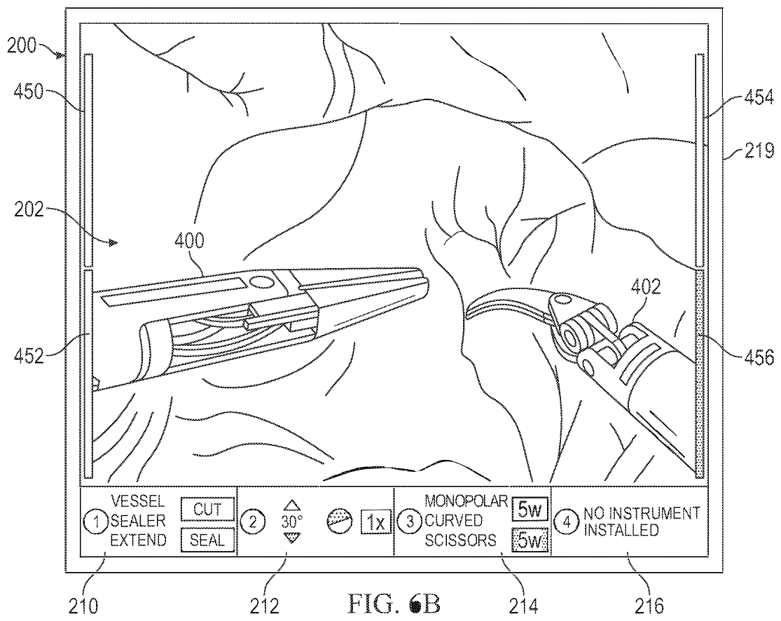

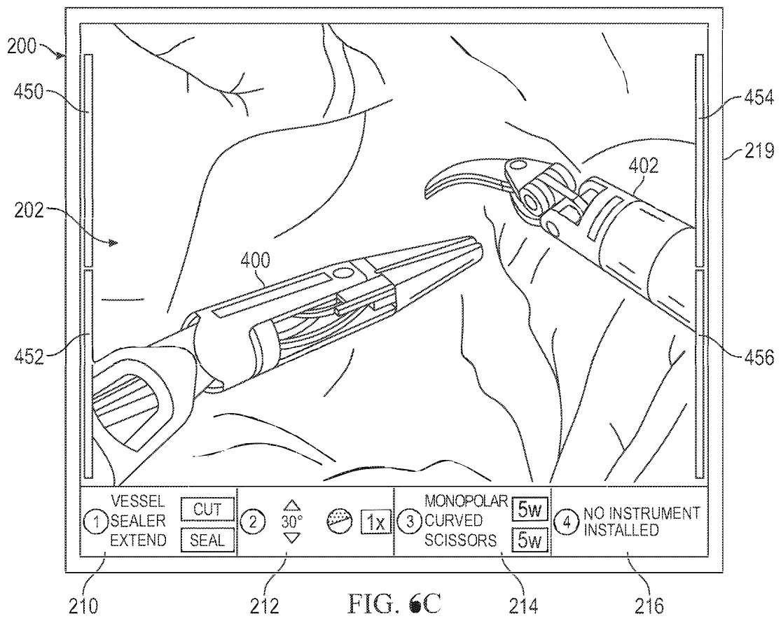

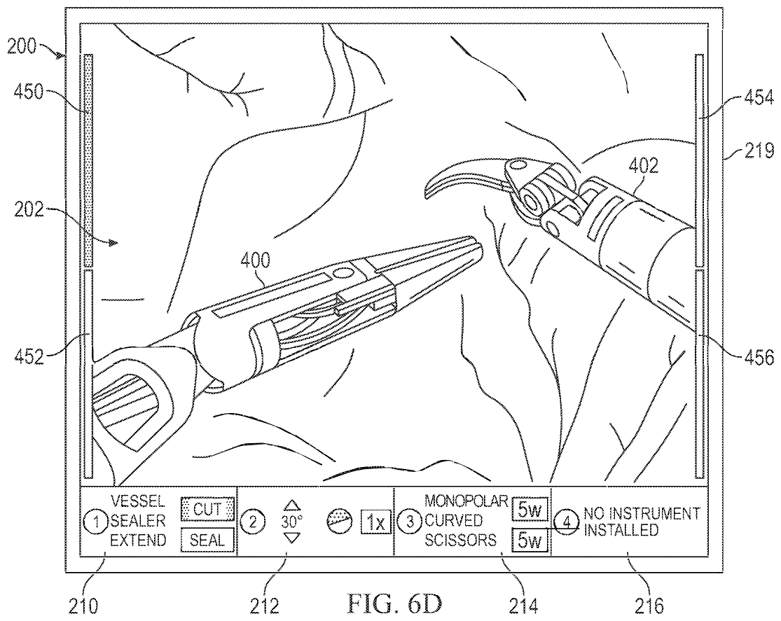

Various types, shapes, and configurations of synthetic indicators may be displayed to provide information about the status of user input device 102 engagement. In an alternative implementation, as shown in FIGS. 6A, 6B, 6C, and 6D, the graphical user interface 200 with medical tools 400, 402 is visible in the field of view portion 202. In this implementation, synthetic indicators 450, 452, 454, 456 may take the form of elongated bars that extend along the perimeter 219.

In this example, the synthetic indicators 450-456 are inside the boundary of the perimeter 219, but in alternative implementations may be outside the perimeter 219 of the field of view 202. In this implementation, the synthetic indicator 450, 452 may perform a function similar to synthetic indicator 404 in providing information about the set of user input devices 102b, 102e. As shown in FIG. 6A, when the range sensors 104 and/or sensors of the user input devices 102 detect that an operator's foot is hovering above or otherwise within a threshold distance from the primary user input device 102d, the synthetic indicator 456 is outlined, indicating to the operator that the operator's foot is near the primary user input device 102d. As shown in FIG. 6B, when the operator engages the user input device 102d, the synthetic indicator 456 may become a filled bar to indicate to the operator that the operator's foot has engaged the user input device 102d and the function of the user input device 102d has been initiated. In some implementations, the hover or engaged status of the user input device 102d may be indicated in the information block 214 using the same or similar graphical indicators.

As shown in FIG. 6C, when the range sensors 104 and/or sensors of the user input devices 102 detect that an operator's foot is hovering above or otherwise within a threshold distance from the secondary user input device 102b, the synthetic indicator 450 is outlined, indicating to the operator that the operator's foot is near the user input device 102b. As shown in FIG. 6D, when the operator engages the user input device 102b, the synthetic indicator 456 may become a filled bar to indicate to the operator that the operator's foot has engaged the user input device 102b and the function of the user input device 102b has been initiated. In some implementations, the hover or engaged status of the user input device 102b may be indicated in the information block 210 using the same or similar graphical indicators.

In alternative implementations, audio cues may be provided instead of or in addition to the synthetic indicators to provide instructions or indicate spatial direction (e.g., up/down/left/right) to move the operator's foot into a hover position for a user input device. The system may distinguish between hovering a foot over a pedal vs. actuating the pedal, and there may be distinct visual and audio cues for hover status versus the engaged or actuation status. The system may also depict when a pedal function is valid or invalid. The highlight color may appear in gray when a pedal function is not valid (e.g., when the instrument function cable not plugged in, or the instrument function is not configured).

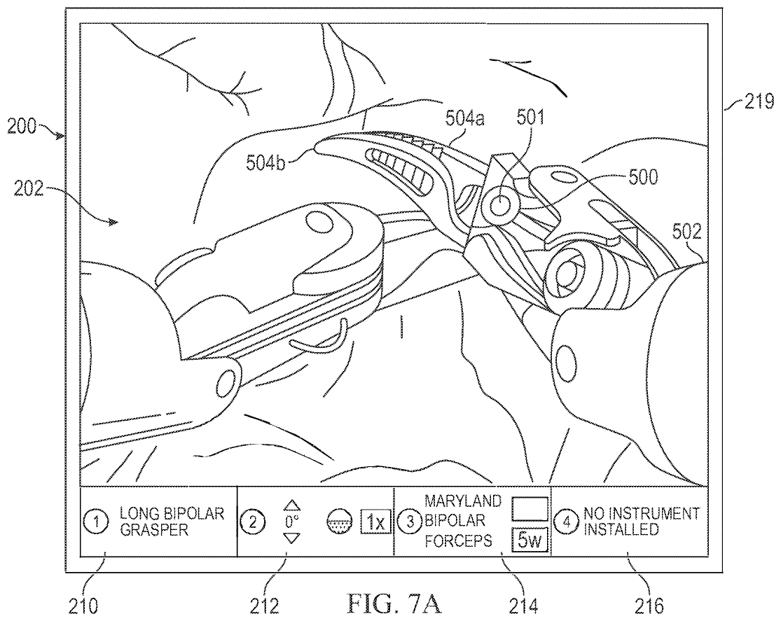

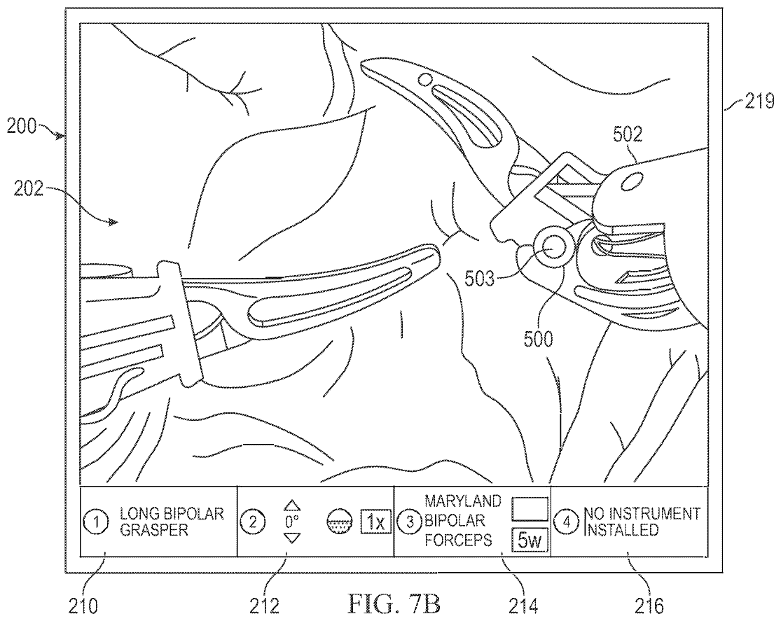

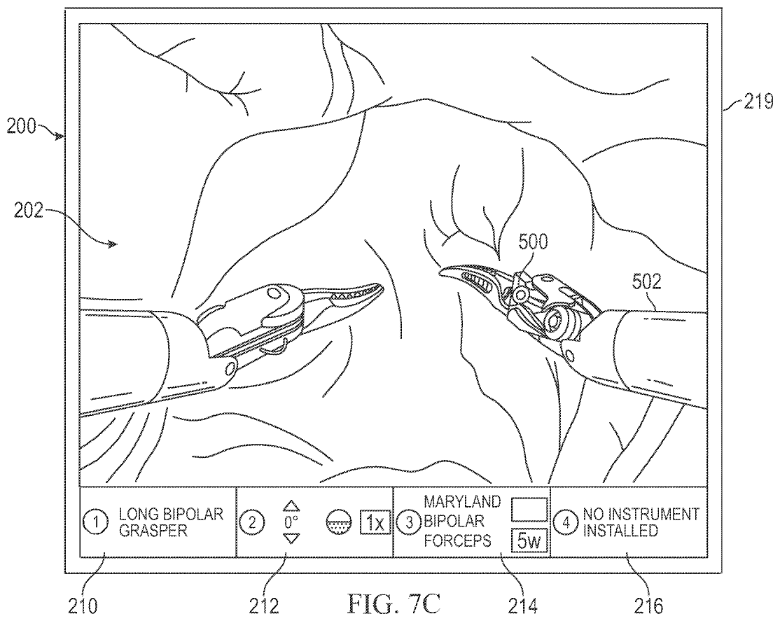

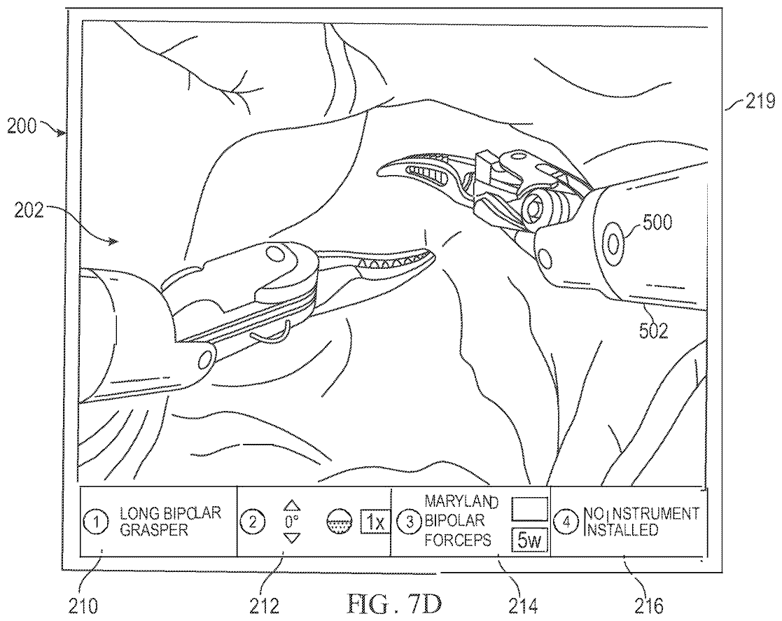

As shown in FIGS. 7A-7D, synthetic indicators that display as badges or labels on components in the field of view portion 202 may appear in proximity to the components and may conditionally move to stay visible and in proximity to the components as the components or the endoscope generating the field of view are moved. Synthetic indicators may be used for any of the purposes described above but may also be used to identify medical tools or other components in the field of view portion 202, identify the manipulator arm to which the medical tool is coupled, provide status information about the medical tool, provide operational information about the medical tool, or provide any other information about the tool or the manipulator arm to which it is coupled.

As shown in FIG. 7A, a synthetic indicator may be associated with a tool 502. In this implementation, the synthetic indicator may be a badge—and is therefore shown as badge 500—configured to have the appearance of a decal on the tool 502. The badge 500 may appear in proximity to jaws 504a, 504b of the tool 502, but may be positioned to avoid occluding the jaws. The placement may include a bias away from the jaws based on the positional uncertainty of the underlying kinematic tracking technology. The default location of the badge 500 may be at a predetermined keypoint 501 on the tool 502. As shown in FIG. 7A, the badge 500 may be placed at a key point 501 located at a clevis of the tool. The badge 500 may pivot and translate as the endoscope or the tool 502 moves so that the badge 500 remains at the keypoint and oriented along a surface of the clevis. When the surface of the clevis is no longer visible in the field of view portion 202, the badge 500 may be moved to another keypoint 503 such as shown in FIG. 7B (at a predetermined joint location) or as shown in FIG. 7D (along the shaft of the tool 502).

The badge 500 may remain at the original keypoint location if the keypoint location remains visible in the field of view portion 202. With reference again to FIG. 7B, because a normal to the badge 500 at the original keypoint (in FIG. 7A) is no longer within the field of view portion 202, the badge 500 may be relocated to a second default keypoint.

The orientation of the badge 500 at a keypoint may be constrained so that the normal to the badge surface is within the field of view portion 202. If the badge 500 may not be oriented at a keypoint such that the normal is within the field of view portion 202, the badge 500 may be moved to a different keypoint. As shown in FIG. 7D, the orientation of the badge 500 may be pivoted to match the orientation of the tool 502 shaft while the surface of the badge 500 remains visible to the viewer. The size of the badge 500 may also change as the distance of the keypoint to which it affixed moves closer or further from the distal end of the endoscope or when a zoom function of the endoscope is activated. The badge size may be governed to stay within maximum and minimum thresholds to avoid becoming too large or too small on the display. As shown in FIG. 7C, the badge 500 may be smaller because the keypoint in FIG. 7C is further from the endoscope than it is in FIG. 7A.

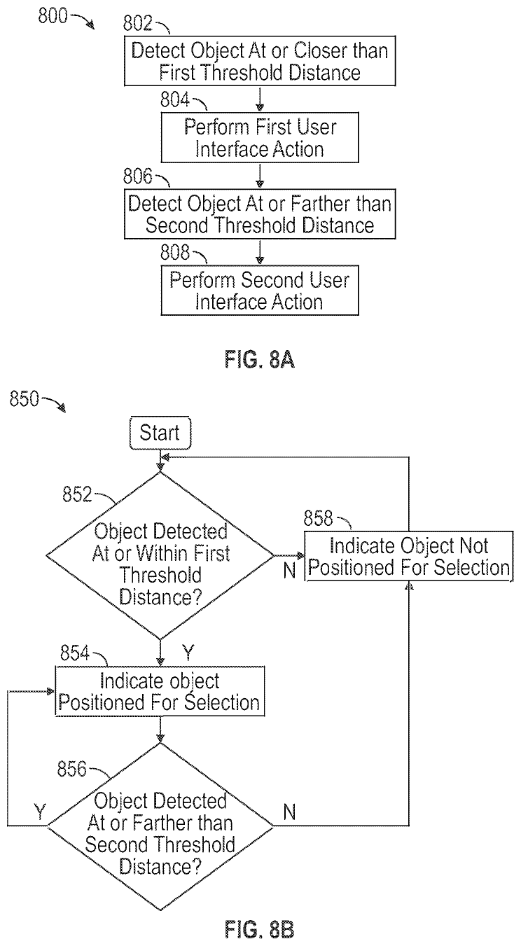

FIG. 8A is a flowchart 800 of operation of the control system 20 according to various implementations described herein. At 802, the control system 20 detects an object at or closer than the first threshold distance 128 for one of the user input devices 102 in the input device tray 100. For example, the range sensor 104 associated with the one of the user input devices measures a distance of an object within the field of view 126 of the range sensor 104. The range sensor 104 is positioned with the field of view 126 across the selection surface 120 of the user input device 102. The control system 20 compares the measured distance from the range sensor 104 with the first threshold distance 128 to determine whether an object is at or within the first threshold distance 128 (e.g., determine if the measured distance is less than or equal to the first threshold distance 128).

At 804, the control system 20 performs the first user interface action to provide feedback to the surgeon S that an object is over or otherwise positioned to facilitate selection of one of the user input devices 102. The first user interface action may be to provide visual feedback (e.g., via display system 35), audio feedback, and/or haptic feedback such as described in the examples provided above.

At 806, the control system 20 detects the object at or farther than the second threshold distance 130 for the user input device 102. The second threshold distance 130 is greater than the first threshold distance 128. For example, the range sensor 104 associated with the user input device 102 measures a distance to the object within the field of view 126 of the range sensor 104. The control system 20 compares the measured distance from the range sensor 104 with the second threshold distance 130 to determine whether the object is at or farther than the second threshold distance 130 (e.g., determine if the measured distance is more than or equal to the second threshold distance 130).

At 808, the control system 20 performs the second user interface action to provide feedback to the surgeon S that the object is no longer over or otherwise positioned to facilitate selection of the user input device 102. The second user interface action may be to discontinue to provide visual feedback (e.g., via display system 35), provide another audio feedback, and/or provide another haptic feedback such as described in the examples provided above.

FIG. 8B is a flowchart 850 of an example threshold-based hysteresis of the control system 20 according to various implementations described herein. At 852, the control system 20 determines whether an object is detected at or within the first threshold distance 128 for one of the user input devices 102 in the input device tray 100. For example, the range sensor 104 associated with the one of the user input devices measures a distance of an object within the field of view 126 of the range sensor 104. The range sensor 104 is positioned with the field of view 126 across the selection surface 120 of the user input device 102. The control system 20 compares the measured distance from the range sensor 104 with the first threshold distance 128 to determine whether an object is at or within the first threshold distance 128 (e.g., determine if the measured distance is less than or equal to the first threshold distance 128).

If an object is detected at or within the first threshold distance 128 at 852, the control system 20 proceeds to 854. At 854, the control system 20 indicates that an object is positioned for selection. For example, the control system 20 performs the first user interface action to provide feedback to the surgeon S that an object is over or otherwise positioned to facilitate selection of one of the user input devices 102. The first user interface action may be to provide visual feedback (e.g., via display system 35), audio feedback, and/or haptic feedback such as described in the examples provided above.

At 806, the control system 20 detects the object at or farther than the second threshold distance 130 for the user input device 102. The second threshold distance 130 is greater than the first threshold distance 128. For example, the range sensor 104 associated with the user input device 102 measures a distance to the object within the field of view 126 of the range sensor 104. The control system 20 compares the measured distance from the range sensor 104 with the second threshold distance 130 to determine whether the object is at or farther than the second threshold distance 130 (e.g., determine if the measured distance is more than or equal to the second threshold distance 130).

If an object is not detected at or within the first threshold distance 128 at 852, the control system 20 proceeds to 858. At 858, the control system 20 indicates an object is not positioned for selection. For example, the control system may not perform any action when transitioning from 852 to 858. Alternatively or additionally, the control system 20 actively indicates that an object is not positioned for selection.

At 808, the control system 20 performs the second user interface action to provide feedback to the surgeon S that the object is no longer over or otherwise positioned to facilitate selection of the user input device 102. The second user interface action may be to discontinue to provide visual feedback (e.g., via display system 35), provide another audio feedback, and/or provide another haptic feedback such as described in the examples provided above.

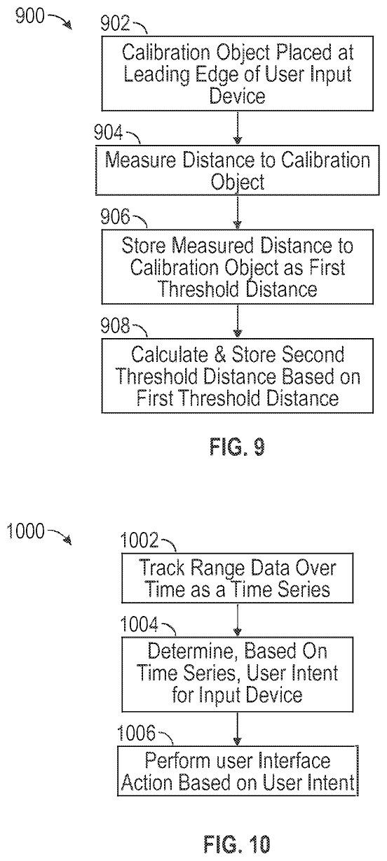

FIG. 9 is a flowchart of a calibration operation 900 according to various implementations described herein. At 902, a calibration object is placed within the field of view 126 of one or more of the range sensors 104. The calibration object is placed along the leading edge 118 of the user input device 102 so that the calibration object is within the field of view 126 at the first threshold distance 128. In various implementations, the calibration object is selected to have a reflectivity similar to or characteristic of an object to be used to select the user input devices 102. For example, the calibration object is selected to have a reflectivity similar to or characteristic of a shoe when the user input devices 102 are foot pedals. Alternatively or additionally, sensor thresholds for the range sensors 104 may be adjusted based on a reflectivity of a shoe worn by the surgeon S. In some implementations, the calibration object may be a given surgeon's shoe.

At 904, the range sensor 104 for the user input device 102 measures a distance to the calibration object. For example, the range sensor 104 may generate a signal indicative of the distance to the calibration object (e.g., time signal, signal intensity value, etc.) and/or may generate a measured distance value (e.g., 75 mm). The control system 20 receives the signal indicative of the distance and/or the measured distance value from the range sensor.

In various implementations, the control system 20 receives a plurality of such distance measurements during a calibration operation. The control system 20 them performs an average, median, mean or other statistical evaluation of the received range data to determine the measured distance to the calibration object.

At 906, the control system 20 stores the measured distance to the calibration object as the first threshold distance 128 for the user input device 102. At 908, the control system 20 calculates and stores the second threshold distance 130 based on the first threshold distance 128. For example, the control system 20 adds the predetermined distance 132 to the first threshold distance 128 to determine the second threshold distance 130.

While the calibration operation 900 is described above for one of the user input devices 102, the calibration operation 900 may be repeated for each of the user input devices 102 in the user input tray 100.

FIG. 10 is a flowchart of a user intent determination 1000 according to various implementations described herein. At 1002, the control system 20 tracks range data of one or more of the range sensors 104 over time as one or more time series of range data.

At 1004, the control system 20 evaluates the time series to determine a user intent with respect to one or more of the user input devices 102. For example, the control system 20 may determine a direction of movement and/or velocity of an object with respect to one or more of the user input devices 102 based on the time series. In some implementations, the control system 20 resolves a three dimensional position, direction of movement, and/or velocity of an object. Movements above a first threshold velocity and/or in a direction away from the user input devices 102 may be determined as an intent to not select one of the user input devices 102. In contrast, movements below a second threshold velocity and/or in a direction toward the user input device may be determined as an intent to select one of the user input devices.

At 1006, the control system 20 performs a user interface action based on the determined user intent. For example, the control system 20 may ignore (e.g., for a predetermined period of time) or otherwise require verification from the surgeon S for any hover or selection events upon a determination of an intent to not select one of the user input devices 102. Alternatively or additionally, the control system 20 may animate or otherwise modify a displayed graphical user interface to provide an indication of the position, direction of movement, and/or velocity of an object. Alternatively or additionally, the control system 20 may initiate one or more control actions associated with the user input devices 102 (e.g., actions that need lead time, change power state of medical tools). Alternatively or additionally, the control system 20 may provide auditory or haptic feedback to the surgeon S.

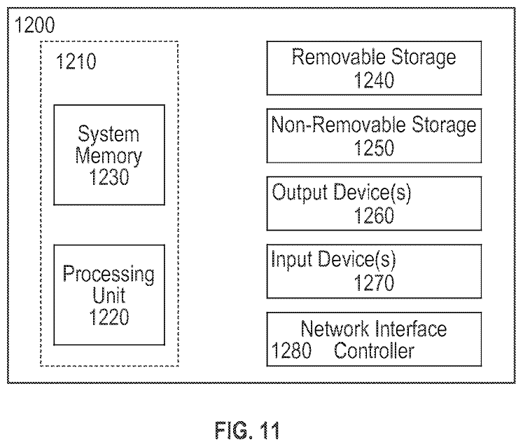

It should be appreciated that the logical operations described herein with respect to the various figures may be implemented (1) as a sequence of computer implemented acts or program modules (i.e., software) running on a computing device (e.g., the computing device described in FIG. 11), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 11, an example computing device 1200 upon which implementations of the invention may be implemented is illustrated. For example, a computer processor located on medical system 10, assembly 12, operator input system 16, control system 20, or auxiliary systems 26 described herein may each be implemented as a computing device, such as computing device 1200. It should be understood that the example computing device 1200 is only one example of a suitable computing environment upon which implementations of the invention may be implemented. Optionally, the computing device 1200 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In an implementation, the computing device 1200 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an implementation, virtualization software may be employed by the computing device 1200 to provide the functionality of a number of servers that is not directly bound to the number of computers in the computing device 1200. For example, virtualization software may provide twenty virtual servers on four physical computers. In an implementation, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In its most basic configuration, computing device 1200 typically includes at least one processing unit 1220 and system memory 1230. Depending on the exact configuration and type of computing device, system memory 1230 may be volatile (such as random-access memory (RAM)), non-volatile (such as read-only memory (ROM), flash memory, etc.), or some combination of the two. This most basic configuration is illustrated in FIG. 11 by dashed line 1210. The processing unit 1220 may be a standard programmable processor that performs arithmetic and logic operations necessary for operation of the computing device 1200. While only one processing unit 1220 is shown, multiple processors may be present. Thus, while instructions may be discussed as executed by a processor, the instructions may be executed simultaneously, serially, or otherwise executed by one or multiple processors. The computing device 1200 may also include a bus or other communication mechanism for communicating information among various components of the computing device 1200.

Computing device 1200 may have additional features/functionality. For example, computing device 1200 may include additional storage such as removable storage 1240 and non-removable storage 1250 including, but not limited to, magnetic or optical disks or tapes. Computing device 1200 may also contain network connection(s) 1280 that allow the device to communicate with other devices such as over the communication pathways described herein. The network connection(s) 1280 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing device 1200 may also have input device(s) 1270 such as a keyboard, keypads, switches, dials, mice, track balls, touch screens, voice recognizers, card readers, paper tape readers, or other well-known input devices. Output device(s) 1260 such as a printer, video monitors, liquid crystal displays (LCDs), touch screen displays, displays, speakers, etc. may also be included. The additional devices may be connected to the bus in order to facilitate communication of data among the components of the computing device 1200. All these devices are well known in the art and need not be discussed at length here.

The processing unit 1220 may be configured to execute program code encoded in tangible, computer-readable media. Tangible, computer-readable media refers to any media that is capable of providing data that causes the computing device 1200 (i.e., a machine) to operate in a particular fashion. Various computer-readable media may be utilized to provide instructions to the processing unit 1220 for execution. Example tangible, computer-readable media may include, but is not limited to, volatile media, non-volatile media, removable media and non-removable media implemented in any method or technology for storage of information such as computer readable instructions, data structures, program modules or other data. System memory 1230, removable storage 1240, and non-removable storage 1250 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware, for example in an application specific integrated circuit (ASIC), because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

In an example implementation, the processing unit 1220 may execute program code stored in the system memory 1230. For example, the bus may carry data to the system memory 1230, from which the processing unit 1220 receives and executes instructions. The data received by the system memory 1230 may optionally be stored on the removable storage 1240 or the non-removable storage 1250 before or after execution by the processing unit 1220.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language and it may be combined with hardware implementations.

Implementations of the methods and systems may be described herein with reference to block diagrams and flowchart illustrations of methods, systems, apparatuses, and computer program products. It will be understood that each block of the block diagrams and flowchart illustrations, and combinations of blocks in the block diagrams and flowchart illustrations, respectively, can be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

While several implementations have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various implementations as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

1-54. (canceled)

55. An object detection and visual feedback system, comprising:
a button positioned for selection by an object, the button comprising a selection surface;
a range sensor with a field of view across the selection surface of the button, wherein the range sensor is configured to measure a distance of an object within the field of view; and
a user interface configured to perform a first action upon the distance measured by the range sensor being less than or equal to a first threshold distance, wherein the user interface is further configured to perform a second action upon the distance measured by the range sensor being greater than or equal to a second threshold distance, wherein the second threshold distance is greater than the first threshold distance.

56. The system of claim 55, wherein the first threshold distance is at an edge of the button.

57. The system of claim 56, wherein the second threshold distance is not coextensive with the button and spaced apart from the edge of the button by a predetermined distance.

58. The system of claim 57, wherein the predetermined distance is sufficient to mitigate against accidental selection of the button by the object, and wherein the predetermined distance is in a range of distances from 10 mm to 30 mm.

59. The system of claim 55, wherein the object is any one of a non-hand limb, a foot, a leg, a knee, a head, an elbow, or an extension from human anatomy, and wherein the range sensor is any of a time-of-flight distance sensor, a triangulation distance sensor, an optical distance sensor, an acoustic distance sensor, an inductive distance sensor, a capacitive distance sensor, a photoelectric distance sensor, a camera, an infrared distance sensor, a laser range finder, or a light detection and ranging sensor.

60. The system of claim 55, wherein a direction of the field of view is parallel to a direction of the distance measured by the range sensor.

61. The system of claim 55, wherein a direction of the field of view is orthogonal to a direction of the distance measured by the range sensor.

62. The system of claim 55, further comprising:
a second button positioned for selection by an object, the second button comprising a second selection surface; and
a second range sensor with a second field of view across the second selection surface of the second button, wherein the second range sensor is configured to measure a distance of an object within the second field of view,
wherein the user interface is configured to perform a third action upon the distance measured by the second range sensor being less than or equal to a third threshold distance.

63. The system of claim 62, wherein the third action is different than the first action.

64. The system of claim 55, wherein the first action is to display an indication of the button.

65. The system of claim 64, wherein the second action is to discontinue display of the indication of the button.

66. The system of claim 64, wherein the indication of the button is selected from a group of indications consisting of an indication of a function performed upon selection of the button; a change of intensity of an icon displayed on the user interface; and a graphic of the object relative to a layout of selection buttons, the layout including the button.

67. The system of claim 55, wherein the first action is to sound a first audible alert and the second action is to sound a second audible alert.

68. The system of claim 55, wherein the first action is to provide a first haptic feedback and the second action is to provide a second haptic feedback.

69. The system of claim 68, wherein the first haptic feedback and the second haptic feedback is provided to a hand controller.

70. A robotic surgical system, comprising:
a user interface; and
a foot tray comprising:
a button positioned for selection by a foot of a user, the button comprising a selection surface; and
a range sensor positioned with a field of view across the selection surface of the button, wherein the range sensor is configured to measure a distance of an object within the field of view,
wherein the user interface is configured to perform a first action associated with the button upon the distance measured by the range sensor being less than or equal to a first threshold distance, wherein the user interface is further configured to perform a second action associated with the button upon the distance measured by the range sensor being greater than or equal to a second threshold distance, wherein the second threshold distance is greater than the first threshold distance.

71. The robotic surgical system of claim 70, wherein the foot tray is positioned on a base of the robotic surgical system.

72. The robotic surgical system of claim 70, wherein the user interface comprises a display, wherein the robotic surgical system further comprises a headrest, wherein the display is incorporated into the headrest, and wherein the display is a stereoscopic display.

73. A method of providing feedback upon detection of an object, the method comprising:
measuring a distance of an object within a field of view of a range sensor, the range sensor positioned with the field of view across a selection surface of a button, wherein the button is positioned for selection by the object;
performing a first action with a user interface upon the distance measured by the range sensor being less than or equal to a first threshold distance; and
performing a second action with the user interface upon the distance measured by the range sensor being greater than a second threshold distance, wherein the second threshold distance is greater than the first threshold distance.

74. The method of claim 73, further comprising:
measuring a second distance of a second object within a second field of view of a second range sensor, the second range sensor positioned with the second field of view across a second selection surface of a second button, wherein the second button is positioned for selection by the object;
performing a third action with the user interface upon the second distance measured by the range sensor being less than or equal to a third threshold distance; and
performing a fourth action with the user interface upon the second distance measured by the second range sensor being greater than a fourth threshold distance, wherein the fourth threshold distance is greater than the third threshold distance.
